(19) United States
(12) Patent Application Publication
(10) Pub. No.: US 2024/0226574 A9
(43) Pub. Date: Jul. 11, 2024

(54) PARAMETER SELECTION FOR ELECTRICAL STIMULATION THERAPY

(71) Applicant: Medtronic, Inc., Minneapolis, MN (US)

(72) Inventor: Scott A. Hareland, Lino Lakes, MN (US)

(21) Appl. No.: 18/546,564

(22) PCT Filed: Feb. 24, 2022
(86) PCT No.: PCT/US2022/017634

§ 371 Date: Aug. 15, 2023

Related U.S. Application Data
(60) Provisional application No. 63/153,359, filed on Feb. 24, 2021.

(51) Int. Cl. A61N 1/36

(52) U.S. Cl. CPC A61N 1/36175; A61N 1/3606; A61N 1/36178

(57) ABSTRACT

Devices, systems, and techniques are described for selecting parameters for electrical stimulation therapy. For example, device may include processing circuitry configured to determine a first window of time to sense a physiological signal, determine, based on the first window of time, a second window of time for delivering electrical stimulations, and determine, based on a duration of the second window of time, a number of stimulation pulses deliverable during the second window of time at one or more pulse frequencies. The processing circuitry may then output, based on the number of stimulation pulses deliverable during the second window of time, at least one selectable stimulation parameter that at least partially defines the electrical stimulation, wherein the second window of time is adjacent to the first window of time.

This application claims priority to U.S. Provisional Patent Application No. 63/153,359, filed Feb. 24, 2021, the entire contents of which is incorporated herein by reference.

### TECHNICAL FIELD

This disclosure generally relates to electrical stimulation therapy, and more specifically, parameter selection for electrical stimulation therapy.

### BACKGROUND

Medical devices may be external or implanted and may be used to deliver electrical stimulation therapy to patients via various tissue sites to treat a variety of symptoms or conditions such as chronic pain, tremor, Parkinson's disease, epilepsy, urinary or fecal incontinence, sexual dysfunction, obesity, or gastroparesis. A medical device may deliver electrical stimulation therapy via one or more leads that include electrodes located proximate to target locations associated with the brain, the spinal cord, pelvic nerves, peripheral nerves, or gastrointestinal tract of a patient. Stimulation proximate the spinal cord, proximate the sacral nerve, within the brain, and proximate peripheral nerves are often referred to as spinal cord stimulation (SCS), sacral neuromodulation (SNM), deep brain stimulation (DBS), and peripheral nerve stimulation (PNS), respectively.

Electrical stimulation therapy may be delivered by the medical device as a train of electrical stimulation pulses, and parameters that define the electrical stimulation pulses may include a frequency, an amplitude, a pulse width, and a pulse shape.

### SUMMARY

In general, this disclosure is directed to devices, systems, and techniques for selecting parameters for electrical stimulation therapy. For example, a system may analyze various parameters that fit within certain constraints on therapy and identify possible parameter values that may be selected to define electrical stimulation therapy. This processing may, in some cases, increase the number of electrical stimulation pulses that can be delivered within a particular window of time.

In some examples, an example method of this disclosure may determine, through processing circuitry, a first window of time for sensing a physiological signal. The example method may then determine, through processing circuitry and based on the first window of time, a second window of time for delivering electrical stimulation. The example method may then determine, based on a duration of the second window of time, a number of stimulation pulses deliverable during the second window of time at one or more pulse frequencies. The example method may then output, based on the number of stimulation pulses deliverable during the second window of time, at least one selectable stimulation parameter that at least partially defines the electrical stimulation. In some examples, the second window of time may be adjacent to the first window of time.

In some examples, an example device of this disclosure may comprise processing circuitry configured to determine a first window of time to sense a physiological signal and determine, based on the first window of time, a second window of time for delivering electrical stimulations. The processing circuitry of the example device may be further configured to determine, based on a duration of the second window of time, a number of stimulation pulses deliverable during the second window of time at one or more pulse frequencies. The processing circuitry of the example device may be further configured to output, based on the number of stimulation pulses deliverable during the second window of time, at least one selectable stimulation parameter that at least partially defines the electrical stimulation. In some examples, the second window of time may be adjacent to the first window of time.

In some examples, an example system of this disclosure may comprise an implanted medical device, a medical device programmer, and processing circuitry configured to determine a first window of time to sense a physiological signal. The processing circuitry of the example system may be further configured to determine, based on the first window of time, a second window of time for delivering electrical stimulations. The processing circuitry of the example system may be further configured to determine, based on a duration of the second window of time, a number of stimulation pulses deliverable during the second window of time at one or more pulse frequencies. The processing circuitry of the example system may be further configured to output, based on the number of stimulation pulses deliverable during the second window of time, at least one selectable stimulation parameter that at least partially defines the electrical stimulation. In some examples, the second window of time is adjacent to the first window of time.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a conceptual diagram illustrating an example system that includes a medical device programmer and an implanted medical device (IMD) configured to deliver spinal cord stimulation (SCS) therapy according to the techniques of the disclosure.

FIG. 2 is a block diagram of the example IMD of FIG. 1.

FIG. 3 is a block diagram of the example medical device programmer of FIG. 1.

FIG. 4A is a flow diagram of an example method of parameter selection for electrical stimulation therapy.

FIG. 4B is a flow diagram of an example method of parameter selection for electrical stimulation therapy, further comprising validating the selectable stimulation parameters and delivering an electrical stimulation therapy at least partially defined by the selectable stimulation parameters.

FIG. 5 is a flow diagram of an example method of determining the selectable stimulation parameters of FIG. 4A.

FIG. 6 is a flow diagram of an example method of validating the selectable stimulation parameters of FIG. 4B.

FIG. 7 is a flow diagram of an example method of delivering stimulation therapies of FIG. 4B.

FIG. 8 is a flow diagram of an example method of determining a selectable stimulation parameter of FIG. 4A based on a selected therapy frequency.

FIG. 9 is a flow diagram of an example method of determining a selectable stimulation parameter of FIG. 4A based on a range of therapy frequencies.

FIG. 10 is a flow diagram of an example method of determining updated system parameters of FIG. 6.

FIG. 11 is a flow diagram of an example method of adjusting a value of the selectable stimulation parameters of FIG. 6.

FIG. 12 is a timing diagram illustrating an example of evoked compound action potential (ECAP)-control intervals of control pulses and respective sensed ECAPs.

FIG. 13 is a timing diagram illustrating an example of stimulation pulses and accompanying phases of an inter-pulse interval.

FIG. 14 is a timing diagram illustrating an example of control pulses, respective sensed ECAPs, and stimulation pulses between adjacent ECAP-control intervals.

FIG. 15 is a graph illustrating the electrical stimulation options of an optimized therapy and an unoptimized therapy at varying therapy frequencies.

FIG. 16 is a flow diagram of an example method for determining the number of stimulation pulses that can be delivered between sensing windows.

FIGS. 17-23 are flow diagrams illustrating portions of the method of FIG. 16.

### DETAILED DESCRIPTION

Systems, devices, and techniques are described herein for improving pulse delivery options for a given window of time within which pulses can be delivered. In some systems, electrical stimulation can be delivered continuously or as desired to treat a patient. In other systems, the electrical stimulation may need to be withheld or stopped for a certain period of time due to various physiological events or system requirements to perform other activities. For example, the system may be configured to sense for various signals (e.g., evoked compound action potentials (ECAPs) or other physiological signals), and electrical stimulation delivered during these sensing windows would impair the ability of the system to detect the desired signals. Therefore, the system may only be able to deliver stimulation pulses during a therapy window between consecutive sensing windows, for example. The limited duration of time within the therapy window may reduce the number of programming options, such as how many pulses can be delivered during the therapy window.

As described herein, systems, devices, and techniques are described for increasing the number of programming options available within a given therapy window. For example, a system may analyze various parameters that fit within certain constraints on therapy and identify possible parameter values that may be selected to define electrical stimulation therapy. This processing may, in some cases, increase the number of electrical stimulation pulses that can be delivered within a particular window of time. The system may also adjust aspects of one or more pulses, such as the type of recharge phase or other timing aspects of one or more pulses in order to increase the number of pulses that the system can deliver within the available therapy window.

The techniques of this disclosure may provide one or more advantages. For example, an example therapy may provide additional flexibility in the number of stimulation pulses that will be delivered to a patient. For example, the system may be capable of delivering more pulses within a therapy window and/or use a greater variation of pulse frequencies. In this manner, the process described herein may allow for more system choice or user choice in selecting stimulation parameter values that define an electrical stimulation therapy.

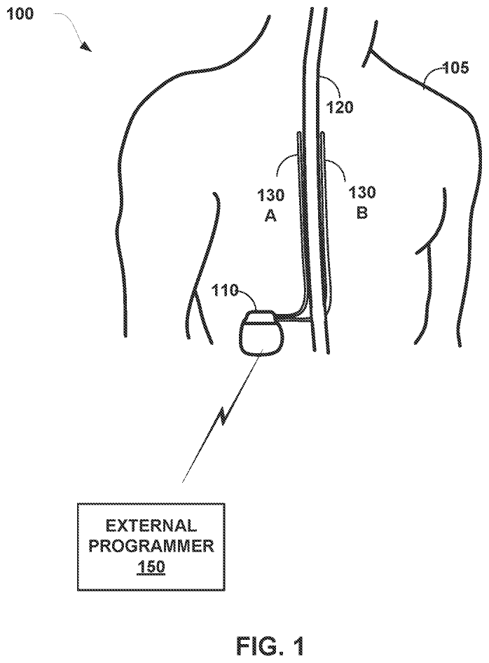

FIG. 1 is a conceptual diagram illustrating an example system 100 that includes an implantable medical device (IMD) 110 configured to deliver spinal cord stimulation (SCS) therapy and an external programmer 150, in accordance with one or more techniques of this disclosure. Although the techniques described in this disclosure are generally applicable to a variety of medical devices including external devices and IMDs, application of such techniques to IMDs and, more particularly, implantable electrical stimulators (e.g., neurostimulators) will be described for purposes of illustration. More particularly, the disclosure will refer to an implantable SCS system for purposes of illustration, but without limitation as to other types of medical devices or other therapeutic applications of medical devices.

As shown in FIG. 1, system 100 includes an IMD 110, leads 130A and 130B, and external programmer 150 shown in conjunction with a patient 105, who is ordinarily a human patient. In the example of FIG. 1, IMD 110 is an implantable electrical stimulator that is configured to generate and deliver electrical stimulation therapy to patient 105 via one or more electrodes of electrodes of leads 130A and/or 130B (collectively, “leads 130”), e.g., for relief of chronic pain or other symptoms. In other examples, IMD 110 may be coupled to a single lead carrying multiple electrodes or more than two leads each carrying multiple electrodes. In some examples, the stimulation signals, or pulses, may be configured to elicit detectable ECAP signals that IMD 110 may use to determine the posture state occupied by patient 105 and/or determine how to adjust one or more parameters that define stimulation therapy. IMD 110 may be a chronic electrical stimulator that remains implanted within patient 105 for weeks, months, or even years. In other examples, IMD 110 may be a temporary, or trial, stimulator used to screen or evaluate the efficacy of electrical stimulation for chronic therapy. In one example, IMD 110 is implanted within patient 105, while in another example, IMD 110 is an external device coupled to percutaneously implanted leads. In some examples, IMD 110 uses one or more leads, while in other examples, IMD 110 is leadless.

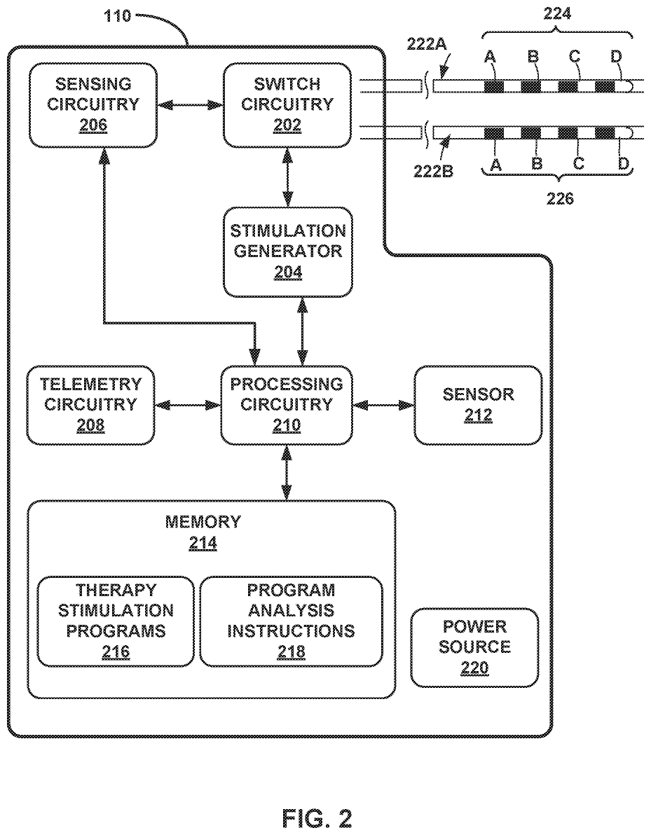

IMD 110 may be constructed of any polymer, metal, or composite material sufficient to house the components of IMD 110 (e.g., components illustrated in FIG. 2) within patient 105. In this example, IMD 110 may be constructed with a biocompatible housing, such as titanium or stainless steel, or a polymeric material such as silicone, polyurethane, or a liquid crystal polymer, and surgically implanted at a site in patient 105 near the pelvis, abdomen, or buttocks. In other examples, IMD 110 may be implanted within other suitable sites within patient 105, which may depend, for example, on the target site within patient 105 for the delivery of electrical stimulation therapy. The outer housing of IMD 110 may be configured to provide a hermetic seal for components, such as a rechargeable or non-rechargeable power source. In addition, in some examples, the outer housing of IMD 110 is selected from a material that facilitates receiving energy to charge the rechargeable power source.

Electrical stimulation energy, which may be constant current or constant voltage-based pulses, for example, is delivered from IMD 110 to one or more target tissue sites of patient 105 via one or more electrodes (not shown) of implantable leads 130. In the example of FIG. 1, leads 130 carry electrodes that are placed adjacent to the target tissue of spinal cord 120. One or more of the electrodes may be disposed at a distal tip of a lead 130 and/or at other positions at intermediate points along the lead. Leads 130 may be implanted and coupled to IMD 110. The electrodes may transfer electrical stimulation generated by an electrical stimulation generator in IMD 110 to tissue of patient 105. Although leads 130 may each be a single lead, lead 130 may include a lead extension or other segments that may aid in implantation or positioning of lead 130. In some other examples, IMD 110 may be a leadless stimulator with one or more arrays of electrodes arranged on a housing of the stimulator rather than leads that extend from the housing. In addition, in some other examples, system 100 may include one lead or more than two leads, each coupled to IMD 110 and directed to similar or different target tissue sites.

The electrodes of leads 130 may be electrode pads on a paddle lead, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for therapy. Ring electrodes arranged at different axial positions at the distal ends of lead 130 will be described for purposes of illustration.

The deployment of electrodes via leads 130 is described for purposes of illustration, but arrays of electrodes may be deployed in different ways. For example, a housing associated with a leadless stimulator may carry arrays of electrodes, e.g., rows and/or columns (or other patterns), to which shifting operations may be applied. Such electrodes may be arranged as surface electrodes, ring electrodes, or protrusions. As a further alternative, electrode arrays may be formed by rows and/or columns of electrodes on one or more paddle leads. In some examples, electrode arrays include electrode segments, which are arranged at respective positions around a periphery of a lead, e.g., arranged in the form of one or more segmented rings around a circumference of a cylindrical lead. In other examples, one or more of leads 130 are linear leads having eight ring electrodes along the axial length of the lead. In another example, the electrodes are segmented rings arranged in a linear fashion along the axial length of the lead and at the periphery of the lead.

The stimulation parameter set of a therapy stimulation program that defines the stimulation pulses of electrical stimulation therapy by IMD 110 through the electrodes of leads 130 may include information identifying which electrodes have been selected for delivery of stimulation according to a stimulation program, the polarities of the selected electrodes, i.e., the electrode combination for the program, voltage or current amplitude, pulse frequency, pulse width, pulse shape of stimulation delivered by the electrodes. These stimulation parameters values that make up the stimulation parameter set that defines pulses may be predetermined parameter values defined by a user and/or automatically determined by system 100 based on one or more factors or user input.

Although FIG. 1 is directed to SCS therapy, e.g., used to treat pain, in other examples system 100 may be configured to treat any other condition that may benefit from electrical stimulation therapy. For example, system 100 may be used to treat tremor, Parkinson's disease, epilepsy, a pelvic floor disorder (e.g., urinary incontinence or other bladder dysfunction, fecal incontinence, pelvic pain, bowel dysfunction, or sexual dysfunction), obesity, gastroparesis, or psychiatric disorders (e.g., depression, mania, obsessive compulsive disorder, anxiety disorders, and the like). In this manner, system 100 may be configured to provide therapy taking the form of deep brain stimulation (DBS), peripheral nerve stimulation (PNS), peripheral nerve field stimulation (PNFS), cortical stimulation (CS), pelvic floor stimulation, gastrointestinal stimulation, or any other stimulation therapy capable of treating a condition of patient 105.

In some examples, lead 130 includes one or more sensors configured to allow IMD 110 to monitor one or more parameters of patient 105, such as patient activity, pressure, temperature, or other characteristics. The one or more sensors may be provided in addition to, or in place of, therapy delivery by lead 130.

IMD 110 is configured to deliver electrical stimulation therapy to patient 105 via selected combinations of electrodes carried by one or both of leads 130, alone or in combination with an electrode carried by or defined by an outer housing of IMD 110. The target tissue for the electrical stimulation therapy may be any tissue affected by electrical stimulation, which may be in the form of electrical stimulation pulses or continuous waveforms. In some examples, the target tissue includes nerves, smooth muscle, or skeletal muscle. In the example illustrated by FIG. 1, the target tissue is tissue proximate spinal cord 120, such as within an intrathecal space or epidural space of spinal cord 120, or, in some examples, adjacent nerves that branch off spinal cord 120. Leads 130 may be introduced into spinal cord 120 in any suitable region, such as the thoracic, cervical, or lumbar regions. Stimulation of spinal cord 120 may, for example, prevent pain signals from traveling through spinal cord 120 and to the brain of patient 105. Patient 105 may perceive the interruption of pain signals as a reduction in pain and, therefore, efficacious therapy results. In other examples, stimulation of spinal cord 120 may produce paresthesia which may be reduce the perception of pain by patient 105, and thus, provide efficacious therapy results.

IMD 110 is configured to generate and deliver electrical stimulation therapy to a target stimulation site within patient 105 via the electrodes of leads 130 to patient 105 according to one or more therapy stimulation programs. A therapy stimulation program defines values for one or more parameters (e.g., a parameter set) that define an aspect of the therapy delivered by IMD 110 according to that program. For example, a therapy stimulation program that controls delivery of stimulation by IMD 110 in the form of pulses may define values for voltage or current pulse amplitude, pulse width, pulse rate (e.g., pulse frequency), electrode combination, pulse shape, etc. for stimulation pulses delivered by IMD 110 according to that program.

ECAPs are a measure of neural recruitment because each ECAP signal represents the superposition of electrical potentials generated from a population of axons firing in response to an electrical stimulus (e.g., a stimulation pulse). Changes in a characteristic (e.g., an amplitude of a portion of the signal or area under the curve of the signal) of an ECAP signals occur as a function of how many axons have been activated by the delivered stimulation pulse. For a given set of parameter values that define the stimulation pulse and a given distance between the electrodes and target nerve, the detected ECAP signal may have a certain characteristic value (e.g., amplitude).

In some examples, effective stimulation therapy may rely on a certain level of neural recruitment at a target nerve. This effective stimulation therapy may provide relief from one or more conditions (e.g., patient perceived pain) without an unacceptable level of side effects (e.g., overwhelming perception of stimulation).

In some examples, the ECAPs detected by an IMD may be ECAPs elicited by stimulation pulses that may or may not be intended to contribute to therapy of a patient or separate pulses (e.g., control pulses) configured to elicit ECAPs that are detectable by the IMD. Nerve impulses detectable as the ECAP signal travel quickly along the nerve fiber after the delivered stimulation pulse first depolarizes the nerve. If the stimulation pulse delivered by first electrodes has a pulse width that is too long, different electrodes configured to sense the ECAP will sense the stimulation pulse itself as an artifact (e.g., detection of delivered charge itself as opposed to detection of a physiological response to the delivered stimulus) that obscures the lower amplitude ECAP signal. However, the ECAP signal loses fidelity as the electrical potentials propagate from the electrical stimulus because different nerve fibers propagate electrical potentials at different speeds and fibers in the spine contributing to the ECAP are pruned off. Therefore, sensing the ECAP at a far distance from the stimulating electrodes may avoid the artifact caused by a stimulation pulse with a long pulse width, but the ECAP signal may be too small or lose fidelity needed to detect changes to the ECAP signal that occur when the electrode to target tissue distance changes. In other words, the system may not be able to identify, at any distance from the stimulation electrodes, ECAPs from stimulation pulses configured to provide a therapy to the patient. These control pulses configured to elicit detectable ECAP signals, and the ECAP signals, may be delivered and sensed during a therapy window described herein. In some examples, the IMD may deliver informed pulses, or therapy pulses, configured to contribute to therapy of the patient. If an ECAP signal cannot be detected from an informed pulse, the system may use the ECAP signal sensed from the control pulse to inform changes to one or more parameter values that define subsequent informed pulses.

IMD 110 may be configured to deliver stimulation to patient 105 via a combination of electrodes of leads 130, alone or in combination with an electrode carried by or defined by an outer housing of IMD 110 in order to detect ECAP signals. The tissue targeted by the stimulation may be the same or similar tissue targeted by the electrical stimulation therapy, but IMD 110 may deliver stimulation pulses for ECAP signal detection via the same, at least some of the same, or different electrodes.

IMD 110 can deliver stimulation to a target stimulation site within patient 105 via the electrodes of leads 130 according to one or more ECAP stimulation programs to develop a growth curve of the ECAP. The one or more ECAP stimulation programs may be stored in a storage device of IMD 110. Each ECAP program of the one or more ECAP stimulation programs includes values for one or more parameters that define an aspect of the stimulation delivered by IMD 110 according to that program, such as current or voltage amplitude, pulse width, pulse frequency, electrode combination. In some examples, the ECAP stimulation program may also define the number of pules and parameter values for each pulse of multiple pulses within a pulse sweep configured to obtain a plurality of ECAP signals for respective pulses in order to obtain the growth curve that IMD 110 may use to determine an estimated neural threshold of the patient. In some examples, IMD 110 delivers stimulation to patient 105 according to multiple ECAP stimulation programs. Although these functions are described with respect to IMD 110, other devices, such as external programmer 150, may perform these functions such as determining the estimated neural threshold based on the growth curve of ECAP characteristic values.

A user, such as a clinician or patient 105, may interact with a user interface of an external programmer 150 to program IMD 110. Programming of IMD 110 may refer generally to the generation and transfer of commands, programs, or other information to control the operation of IMD 110. In this manner, IMD 110 may receive the transferred commands and programs from external programmer 150 to control stimulation, such as electrical stimulation therapy to develop the growth curve. For example, external programmer 150 may transmit therapy stimulation programs, ECAP stimulation programs, stimulation parameter adjustments, therapy stimulation program selections, ECAP program selections, user input, or other information to control the operation of IMD 110, e.g., by wireless telemetry or wired connection.

In some cases, external programmer 150 may be characterized as a physician or clinician programmer if it is primarily intended for use by a physician or clinician. In other cases, external programmer 150 may be characterized as a patient programmer if it is primarily intended for use by a patient. A patient programmer may be generally accessible to patient 105 and, in many cases, may be a portable device that may accompany patient 105 throughout the patient's daily routine. For example, a patient programmer may receive input from patient 105 when the patient wishes to terminate or change electrical stimulation therapy, when a patient perceives stimulation being delivered or when a patient terminates due to comfort level. In general, a physician or clinician programmer may support selection and generation of programs by a clinician for use by IMD 110, whereas a patient programmer may support adjustment and selection of such programs by a patient during ordinary use. In other examples, external programmer 150 may include, or be part of, an external charging device that recharges a power source of IMD 110. In this manner, a user may program and charge IMD 110 using one device, or multiple devices.

As described herein, information may be transmitted between external programmer 150 and IMD 110. Therefore, IMD 110 and external programmer 150 may communicate via wireless communication using any techniques known in the art. Examples of communication techniques may include, for example, radiofrequency (RF) telemetry and inductive coupling, but other techniques are also contemplated. In some examples, external programmer 150 includes a communication head that may be placed proximate to the patient's body near the IMD 110 implant site to improve the quality or security of communication between IMD 110 and external programmer 150. Communication between external programmer 150 and IMD 110 may occur during power transmission or separate from power transmission.

In some examples, IMD 110, in response to commands from external programmer 150, delivers electrical stimulation therapy according to a plurality of therapy stimulation programs to a target tissue site of the spinal cord 120 of patient 105 via electrodes (not depicted) on leads 130. In some examples, IMD 110 modifies therapy stimulation programs as therapy needs of patient 105 evolve over time. For example, the modification of the therapy stimulation programs may cause the adjustment of at least one parameter of the plurality of therapy pulses. When patient 105 receives the same therapy for an extended period, the efficacy of the therapy may be reduced. In some cases, parameters of the plurality of therapy pulses may be automatically updated. In some examples, IMD 110 may detect ECAP signals from pulses delivered for the purpose of providing therapy to the patient.

In some examples, efficacy of electrical stimulation therapy may be indicated by one or more characteristics of an action potential that is evoked by a stimulation pulse delivered by IMD 110, for example by determining an estimated neural response using the characteristic value of the ECAP signal. Electrical stimulation therapy delivery by leads 130 of IMD 110 may cause neurons within the target tissue to evoke a compound action potential that travels up and down the target tissue, eventually arriving at sensing electrodes of IMD 110. Furthermore, stimulation pulses may also elicit at least one ECAP signal, and ECAPs responsive to stimulation may also be a surrogate for the effectiveness of the therapy and/or the intensity perceived by the patient. The amount of action potentials (e.g., number of neurons propagating action potential signals) that are evoked may be based on the various parameters of electrical stimulation pulses such as amplitude, pulse width, frequency, pulse shape (e.g., slew rate at the beginning and/or end of the pulse), etc. The slew rate may define the rate of change of the voltage and/or current amplitude of the pulse at the beginning and/or end of each pulse or each phase within the pulse. For example, a very high slew rate indicates a steep or even near vertical edge of the pulse, and a low slew rate indicates a longer ramp up (or ramp down) in the amplitude of the pulse. In some examples, these parameters contribute to an intensity of the electrical stimulation. In addition, a characteristic of the ECAP signal (e.g., an amplitude) may change based on the distance between the stimulation electrodes and the nerves subject to the electrical field produced by the delivered control stimulation pulses.

Example techniques for adjusting stimulation parameter values for pulses (e.g., pulses configured to contribute to therapy for the patient) are based on comparing the value of a characteristic of a measured ECAP signal to a target ECAP characteristic value. In some examples, the target ECAP characteristic value may be the estimated neural threshold or a value calculated based on the estimated neural threshold (e.g., a percentage below or above 100% of the estimated neural threshold). During delivery of control stimulation pulses defined by one or more ECAP test stimulation programs, IMD 110, via two or more electrodes interposed on leads 130, senses electrical potentials of tissue of the spinal cord 120 of patient 105 to measure the electrical activity of the tissue. IMD 110 senses ECAPs from the target tissue of patient 105, e.g., with electrodes on one or more leads 130 and associated sense circuitry. In some examples, IMD 110 receives a signal indicative of the ECAP from one or more sensors, e.g., one or more electrodes and circuitry, internal or external to patient 105. Such an example signal may include a signal indicating an ECAP of the tissue of patient 105.

In the example of FIG. 1, IMD 110 is described as performing a plurality of processing and computing functions. However, external programmer 150 instead may perform one, several, or all of these functions. In this alternative example, IMD 110 functions to relay sensed signals to external programmer 150 for analysis, and external programmer 150 transmits instructions to IMD 110 to adjust the one or more parameters defining the electrical stimulation therapy based on analysis of the sensed signals. For example, IMD 110 may relay the sensed signal indicative of an ECAP to external programmer 150. External programmer 150 may compare the parameter value of the ECAP to the target ECAP characteristic value relative to an estimated neural response, and in response to the comparison, external programmer 150 may instruct IMD 110 to adjust one or more stimulation parameters that define the electrical stimulation pulses delivered to patient 105.

In the example techniques described in this disclosure, the stimulation parameters and the target ECAP characteristic values associated with the estimated neural response may be initially set at the clinic but may be set and/or adjusted at home by patient 105. For example, the target ECAP characteristics may be changed to match, be a fraction of, or a multiplier of, a stimulation threshold. In some examples, target ECAP characteristics may be specific to respective different posture states of the patient. Once the target ECAP characteristic values are set, the example techniques allow for automatic adjustment of parameter values that define stimulation pulses to maintain consistent volume of neural activation and consistent perception of therapy for the patient. The ability to change the stimulation parameter values may also allow the therapy to have long term efficacy, with the ability to keep the intensity of the stimulation (e.g., as indicated by the ECAP) consistent by comparing the measured ECAP values to the target ECAP characteristic value. In addition, or alternatively, to maintaining stimulation intensity, IMD 110 may monitor the characteristic values of the ECAP signals to limit one or more parameter values that define stimulation pulses. IMD 110 may perform these changes without intervention by a physician or patient 105.

In some examples, the system changes the target ECAP characteristic value over a period of time, such as according to a change to a stimulation threshold (e.g., a perception threshold or detection threshold). The system may be programmed to change the target ECAP characteristic in order to adjust the intensity of stimulation pulses to provide varying sensations to the patient (e.g., increase or decrease the volume of neural activation). Although the system may change the target ECAP characteristic value, received ECAP signals may still be used by the system to adjust one or more parameter values of the stimulation pulse in order to meet the target ECAP characteristic value.

One or more devices within system 100, such as IMD 110 and/or external programmer 150, may perform various functions as described herein. For example, IMD 110 or programmer 150 may include processing circuitry configured to determine a first window of time to sense a physiological signal, determine, based on the first window of time, a second window of time for delivering electrical stimulations, determine, based on a duration of the second window of time, a number of stimulation pulses deliverable during the second window of time at one or more pulse frequencies. and output, based on the number of stimulation pulses deliverable during the second window of time, at least one selectable stimulation parameter that at least partially defines the electrical stimulation, wherein the second window of time is adjacent to the first window of time.

The processing circuitry may also be configured to determine the number of stimulation pulses that are deliverable by, for each frequency of the one or more frequencies: determining a duration of an inter-pulse interval, determine a number of durations of the inter-pulse interval that fit within the duration of the second window of time, and select the number of stimulation pulses less than or equal to the number of durations of the inter-pulse interval that fit within the duration of the second window of time. The inter-pulse interval may include a stimulus phase, a recharge phase, and an idle phase.

The physiological signal may include one or more evoked compound action potential (ECAP) signals, wherein the first window of time comprises an ECAP control interval, and wherein the second window of time comprises a duration between two adjacent ECAP control intervals. The ECAP control interval may be the sensing window and may include a control pulse configured to elicit an ECAP signal of the one or more ECAP signals.

In some examples, the processing circuitry may be configured to validate the selectable stimulation parameter, wherein the processing circuitry validates the selectable stimulation parameter by iteratively determining a set of updated parameters, determine, based on the set of updated parameters a validation condition, determine a violation of the validation condition, and responsive to determining the violation of the validation condition, adjust a value of the at least one selectable stimulation parameter. In some examples, determining the set of update parameters may include: determining, based on the number of stimulation pulses deliverable during the second window of time, an updated second window of time, and determining, based on the updated second window of time, an updated first window of time. Determining the violation of the validation condition comprises determining that an idle phase of an inter-pulse interval is longer than the updated first window of time.

In some examples, the processing circuitry may adjust the selectable stimulation parameter by at least determining, based on the number of stimulation pulses deliverable during the second window of time, an updated number of stimulation pulses deliverable during the updated second window of time and determining a set of adjusted parameters. The process of determining the set of adjusted parameters may include determining, based on the updated number of stimulation pulses deliverable during the updated second window of time, an adjusted second window of time and determining, based on the adjusted second window of time, an adjusted first window of time.

The processing circuitry may be further configured to deliver the electrical stimulation, wherein the electrical stimulation is at least partially defined by the one or more selectable stimulation parameters. In some examples, the processing circuitry controls an implanted medical device to deliver the electrical stimulation. The processing circuitry may be contained by the housing of a medical device programmer, such as programmer 150.

Although in one example IMD 110 takes the form of an SCS device, in other examples, IMD 110 takes the form of any combination of deep brain stimulation (DBS) devices, peripheral nerve stimulators, implantable cardioverter defibrillators (ICDs), pacemakers, cardiac resynchronization therapy devices (CRT-Ds), left ventricular assist devices (LVADs), implantable sensors, orthopedic devices, or drug pumps, as examples. Moreover, techniques of this disclosure may be used to determine stimulation thresholds (e.g., perception thresholds and detection thresholds) associated with any one of the aforementioned IMDs and then use a stimulation threshold to inform the intensity (e.g., stimulation levels) of therapy.

FIG. 2 is a block diagram illustrating an example combination of components of an IMD 110, in accordance with one or more techniques of this disclosure. IMD 110 may be an example of IMD 110 of FIG. 1. In the example shown in FIG. 2, IMD 110 includes stimulation generator 204, switch circuitry 202, sensing circuitry 206, telemetry circuitry 208, processing circuitry 210, memory 214, sensor(s) 212, and power source 220.

In the example shown in FIG. 2, memory 214 stores therapy stimulation programs 216 and program analysis instructions 218. In some examples, therapy stimulation programs 216 may include stimulation parameter values for respective different stimulation programs selectable by the clinician or patient for therapy and timing of stimulation pulses which may include therapy windows and sensing windows such as for employing ECAP sensing. In this manner, each stored therapy stimulation program defines values for a set of electrical stimulation parameters (e.g., a stimulation parameter set), such as a stimulation electrode combination, electrode polarity, current or voltage amplitude, pulse width, pulse rate, and pulse shape, or duty cycle. Program analysis instructions 218 may include instructions that enable processing circuitry 210 to employ various aspects described herein, such as analyzing the therapy window for available time to deliver pulses, identifying when additional pulses are possible, and providing notifications to the user when additional pulse frequencies, pulse widths, or number of pulses are available for programming.

In some examples, stimulation generator 204 generates electrical stimulation signals in accordance with the electrical stimulation parameters noted above. Other ranges of stimulation parameter values may also be useful and may depend on the target stimulation site within patient 105. While stimulation pulses are described, stimulation signals may be of any form, such as continuous-time signals (e.g., sine waves) or the like. Switch circuitry 202 may include one or more switch arrays, one or more multiplexers, one or more switches (e.g., a switch matrix or other collection of switches), or other electrical circuitry configured to direct stimulation signals from stimulation generator 204 to one or more of electrodes 224, 226, or direct sensed signals from one or more of electrodes 224, 226 to sensing circuitry 206. In other examples, stimulation generator 204 and/or sensing circuitry 206 may include sensing circuitry to direct signals to and/or from one or more of electrodes 224, 226, which may or may not also include switch circuitry 202.

Sensing circuitry 206 is configured to monitor signals from any combination of electrodes 224, 226. In some examples, sensing circuitry 206 includes one or more amplifiers, filters, and analog-to-digital converters. Sensing circuitry 206 may be used to sense physiological signals, such as ECAP signals. In some examples, sensing circuitry 206 detects ECAPs from a particular combination of electrodes 224, 226. In some cases, the particular combination of electrodes for sensing ECAPs includes different electrodes than a set of electrodes 224, 226 used to deliver stimulation pulses. Alternatively, in other cases, the particular combination of electrodes used for sensing ECAPs includes at least one of the same electrodes as a set of electrodes used to deliver stimulation pulses to patient 105. Sensing circuitry 206 may provide signals to an analog-to-digital converter, for conversion into a digital signal for processing, analysis, storage, or output by processing circuitry 210.

Telemetry circuitry 208 supports wireless communication between IMD 110 and an external programmer (not shown in FIG. 2) or another computing device under the control of processing circuitry 210. Processing circuitry 210 of IMD 110 may receive, as updates to programs, values for various stimulation parameters such as amplitude and electrode combination, from the external programmer via telemetry circuitry 208. Processing circuitry 210 may store updates to the stimulation parameter settings of therapy stimulation programs 216 or any other data in memory 214. Telemetry circuitry 208 in IMD 110, as well as telemetry circuits in other devices and systems described herein, such as the external programmer, may accomplish communication by radiofrequency (RF) communication techniques. In addition, telemetry circuitry 208 may communicate with an external medical device programmer (not shown in FIG. 2) via proximal inductive interaction of IMD 110 with the external programmer. The external programmer may be one example of external programmer 150 of FIG. 1. Accordingly, telemetry circuitry 208 may send information to the external programmer on a continuous basis, at periodic intervals, or upon request from IMD 110 or the external programmer.

Processing circuitry 210 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210 herein may be embodied as firmware, hardware, software or any combination thereof. Processing circuitry 210 controls stimulation generator 204 to generate stimulation signals according to stimulation parameter settings of therapy stimulation programs 216 and any other instructions stored in memory 214 to apply stimulation parameter values specified by one or more of programs, such as amplitude, pulse width, pulse rate, and pulse shape of each of the stimulation signals.

In the example shown in FIG. 2, one set of electrodes includes electrodes 224, and another set of electrodes includes electrodes 226. In other examples, a single lead may include 16 electrodes 224 or 226 along a single axial length of the lead. In other examples, a single lead may include more than 8 contacts. In some examples, one or more leads may include electrodes as shown in FIG. 2.

Processing circuitry 210 also controls stimulation generator 204 to generate and apply the stimulation signals to selected combinations of electrodes 224, 226. In some examples, stimulation generator 204 includes a switch circuit (instead of, or in addition to, switch circuitry 202) that may couple stimulation signals to selected conductors within leads 222A and 222B (collectively “leads 222”), which, in turn, deliver the stimulation signals across selected electrodes 224, 226. Such a switch circuit may be a switch array, switch matrix, multiplexer, or any other type of switching circuit configured to selectively couple stimulation energy to selected electrodes 224, 226 and to selectively sense bioelectrical neural signals of a spinal cord of the patient (not shown in FIG. 2) with selected electrodes 224, 226.

In other examples, however, stimulation generator 204 does not include a switch circuit and switch circuitry 202 does not interface between stimulation generator 204 and electrodes 224, 226. In these examples, stimulation generator 204 includes a plurality of pairs of voltage sources, current sources, voltage sinks, or current sinks connected to each of electrodes 224, 226 such that each pair of electrodes has a unique signal circuit. In other words, in these examples, each of electrodes 224, 226 is independently controlled via its own signal circuit (e.g., via a combination of a regulated voltage source and sink or regulated current source and sink), as opposed to switching signals between electrodes 224, 226.

Electrodes 224, 226 on respective leads 222 may be constructed of a variety of different designs. For example, one or both of leads 222 may include one or more electrodes at each longitudinal location along the length of the lead, such as one electrode at different perimeter locations around the perimeter of the lead at each of the locations A, B, C, and D. In one example, the electrodes may be electrically coupled to stimulation generator 204, e.g., via switch circuitry 202 and/or switching circuitry of the stimulation generator 204, via respective wires that are straight or coiled within the housing of the lead and run to a connector at the proximal end of the lead. In another example, each of the electrodes of the lead may be electrodes deposited on a thin film. The thin film may include an electrically conductive trace for each electrode that runs the length of the thin film to a proximal end connector. The thin film may then be wrapped (e.g., a helical wrap) around an internal member to form the lead 222. These and other constructions may be used to create a lead with a complex electrode geometry.

Although sensing circuitry 206 is incorporated into a common housing with stimulation generator 204 and processing circuitry 210 in FIG. 2, in other examples, sensing circuitry 206 may be in a separate housing from IMD 110 and may communicate with processing circuitry 210 via wired or wireless communication techniques. In some examples, one or more of electrodes 224 and 226 are suitable for sensing the ECAPs. For instance, electrodes 224 and 226 may sense the voltage amplitude of a portion of the ECAP signals, where the sensed voltage amplitude, such as the voltage difference between features within the signal, is a characteristic the ECAP signal.

Memory 214 may be configured to store information within IMD 110 during operation. Memory 214 may include a computer-readable storage medium or computer-readable storage device. In some examples, memory 214 includes one or more of a short-term memory or a long-term memory. Memory 214 may include, for example, random access memories (RAM), dynamic random access memories (DRAM), static random access memories (SRAM), magnetic discs, optical discs, flash memories, or forms of electrically programmable memories (EPROM) or electrically erasable and programmable memories (EEPROM).

In some examples, memory 214 may store instructions on how processing circuitry 210 can adjust stimulation pulses in response to the determined characteristic values of ECAP signals. For example, processing circuitry 210 may monitor ECAP characteristic values obtained from ECAP signals (or a signal derived from the ECAP signal) to modulate stimulation parameter values (e.g., increase or decrease stimulation intensity to maintain a target therapeutic effect). In some examples, a target ECAP characteristic value may vary for different situations for a patient, such as different posture states, times of day, activities, etc.

Sensor(s) 212 may include one or more sensing elements that sense values of a respective patient parameter, such as posture state. As described, electrodes 224 and 226 may be the electrodes that sense the characteristic value of the ECAP signal. Sensor(s) 212 may include one or more accelerometers, optical sensors, chemical sensors, temperature sensors, pressure sensors, or any other types of sensors. Sensor(s) 212 may output patient parameter values that may be used as feedback to control delivery of therapy. For example, sensor(s) 212 may indicate patient activity, and processing circuitry 210 may increase the frequency of control pulses and ECAP sensing in response to detecting increased patient activity. In one example, processing circuitry 210 may initiate control pulses and corresponding ECAP sensing in response to a signal from sensor(s) 212 indicating that patient activity has exceeded an activity threshold. Conversely, processing circuitry 210 may decrease the frequency of control pulses and ECAP sensing in response to detecting decreased patient activity. For example, in response to sensor(s) 212 no longer indicating that the sensed patient activity exceeds a threshold, processing circuitry 210 may suspend or stop delivery of control pulses and ECAP sensing. In this manner, processing circuitry 210 may dynamically deliver control pulses and sense ECAP signals based on patient activity to reduce power consumption of the system when the electrode-to-neuron distance is not likely to change and increase system response to ECAP changes when electrode-to-neuron distance is likely to change. IMD 110 may include additional sensors within the housing of IMD 110 and/or coupled via one of leads 130 or other leads. In addition, IMD 110 may receive sensor signals wirelessly from remote sensors via telemetry circuitry 208, for example. In some examples, one or more of these remote sensors may be external to patient (e.g., carried on the external surface of the skin, attached to clothing, or otherwise positioned external to patient 105). In some examples, signals from sensor(s) 212 indicate a position or body state (e.g., sleeping, awake, sitting, standing, or the like), and processing circuitry 210 may select target ECAP characteristic values according to the indicated position or body state.

Power source 220 is configured to deliver operating power to the components of IMD 110. Power source 220 may include a battery and a power generation circuit to produce the operating power. In some examples, the battery is rechargeable to allow extended operation. In some examples, recharging is accomplished through proximal inductive interaction between an external charger and an inductive charging coil within IMD 110. Power source 220 may include any one or more of a plurality of different battery types, such as nickel cadmium batteries and lithium ion batteries.

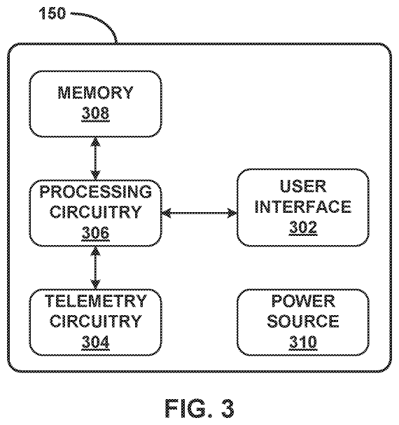

FIG. 3 is a block diagram of the example medical device programmer of FIG. 1. FIG. 3 is a block diagram illustrating an example combination of components of an example external programmer 150. Although external programmer 150 may generally be described as a hand-held device, external programmer 150 may be a larger portable device or a more stationary device. In addition, in other examples, external programmer 150 may be included as part of an external charging device or include the functionality of an external charging device. As illustrated in FIG. 3, external programmer 150 may include processing circuitry 306, memory 308, user interface 302, telemetry circuitry 304, and power source 310. Memory 308 may store instructions that, when executed by processing circuitry 306, cause processing circuitry 306 and external programmer 150 to provide the functionality ascribed to external programmer 150 throughout this disclosure. Each of these components, circuitry, or modules, may include electrical circuitry that is configured to perform some, or all of the functionality described herein. For example, processing circuitry 306 may include processing circuitry configured to perform the processes discussed with respect to processing circuitry 306.

In general, external programmer 150 includes any suitable arrangement of hardware, alone or in combination with software and/or firmware, to perform the techniques attributed to external programmer 150, and processing circuitry 352, user interface 302, and telemetry circuitry 304 of external programmer 150. In various examples, external programmer 150 may include one or more processors, such as one or more microprocessors, DSPs, ASICs, FPGAs, or any other equivalent integrated or discrete logic circuitry, as well as any combinations of such components. External programmer 150 also, in various examples, may include a memory 308, such as RAM, ROM, PROM, EPROM, EEPROM, flash memory, a hard disk, a CD-ROM, including executable instructions for causing the one or more processors to perform the actions attributed to them. Moreover, although processing circuitry 306 and telemetry circuitry 304 are described as separate modules, in some examples, processing circuitry 306 and telemetry circuitry 304 are functionally integrated. In some examples, processing circuitry 306 and telemetry circuitry 304 correspond to individual hardware units, such as ASICs, DSPs, FPGAs, or other hardware units.

Memory 308 (e.g., a storage device) may store instructions that, when executed by processing circuitry 306, cause processing circuitry 306 and external programmer 150 to provide the functionality ascribed to external programmer 150 throughout this disclosure. For example, memory 308 may include instructions that cause processing circuitry 306 to obtain a parameter set from memory, select a spatial electrode pattern, or receive a user input and send a corresponding command to IMD 110, or instructions for any other functionality. In addition, memory 308 may include a plurality of programs, where each program includes a parameter set that defines therapy stimulation or control stimulation. Memory 308 may also store data received from a medical device (e.g., IMD 110). For example, memory 308 may store ECAP related data recorded at a sensing module of the medical device, and memory 308 may also store data from one or more sensors of the medical device.

User interface 302 may include a button or keypad, lights, a speaker for voice commands, a display, such as a liquid crystal (LCD), light-emitting diode (LED), or organic light-emitting diode (OLED). In some examples the display includes a touch screen. User interface 302 may be configured to display any information related to the delivery of electrical stimulation, identified posture states, sensed patient parameter values, or any other such information. User interface 302 may also receive user input (e.g., indication of when the patient perceives a stimulation pulse or which parameters to select) via user interface 302. The input may be, for example, in the form of pressing a button on a keypad or selecting an icon from a touch screen. The input may request starting or stopping electrical stimulation, the input may request a new spatial electrode pattern or a change to an existing spatial electrode pattern, of the input may request some other change to the delivery of electrical stimulation.

Telemetry circuitry 304 may support wireless communication between the medical device and external programmer 150 under the control of processing circuitry 306. Telemetry circuitry 304 may also be configured to communicate with another computing device via wireless communication techniques, or direct communication through a wired connection. In some examples, telemetry circuitry 304 provides wireless communication via an RF or proximal inductive medium. In some examples, telemetry circuitry 304 includes an antenna, which may take on a variety of forms, such as an internal or external antenna.

Examples of local wireless communication techniques that may be employed to facilitate communication between external programmer 150 and IMD 110 include RF communication according to the 802.11 or Bluetooth® specification sets or other standard or proprietary telemetry protocols. In this manner, other external devices may be capable of communicating with external programmer 150 without needing to establish a secure wireless connection. As described herein, telemetry circuitry 304 may be configured to transmit a spatial electrode movement pattern or other stimulation parameter values to IMD 110 for delivery of electrical stimulation therapy. Although IMD 110 may determine characteristic values for ECAP signals and control the adjustment of stimulation parameter values in some examples, programmer 150 may perform these tasks alone or together with IMD 110 in a distributed function.

In some examples, selection of stimulation parameters or therapy stimulation programs are transmitted to the medical device for delivery to a patient (e.g., patient 105 of FIG. 1). In other examples, the therapy may include medication, activities, or other instructions that patient 105 must perform themselves or a caregiver perform for patient 105. In some examples, external programmer 150 provides visual, audible, and/or tactile notifications that indicate there are new instructions. External programmer 150 requires receiving user input acknowledging that the instructions have been completed in some examples.

User interface 302 of external programmer 150 may also be configured to receive an indication from a clinician instructing a processor of the medical device to update one or more therapy stimulation programs or to update the target characteristic values for ECAP signals. Updating therapy stimulation programs and target characteristic values may include changing one or more parameters of the stimulation pulses delivered by the medical device according to the programs, such as amplitude, pulse width, frequency, and pulse shape of the pulses and/or control pulses. User interface 302 may also receive instructions from the clinician commanding any electrical stimulation, including therapy stimulation and control stimulation to commence or to cease.

Power source 310 is configured to deliver operating power to the components of external programmer 150. Power source 310 may include a battery and a power generation circuit to produce the operating power. In some examples, the battery is rechargeable to allow extended operation. Recharging may be accomplished by electrically coupling power source 310 to a cradle or plug that is connected to an alternating current (AC) outlet. In addition, recharging may be accomplished through proximal inductive interaction between an external charger and an inductive charging coil within external programmer 150. In other examples, traditional batteries (e.g., nickel cadmium or lithium ion batteries) may be used. In addition, external programmer 150 may be directly coupled to an alternating current outlet to operate.

The architecture of external programmer 150 illustrated in FIG. 3 is shown as an example. The techniques as set forth in this disclosure may be implemented in the example external programmer 150 of FIG. 3, as well as other types of systems not described specifically herein. Nothing in this disclosure should be construed so as to limit the techniques of this disclosure to the example architecture illustrated by FIG. 3.

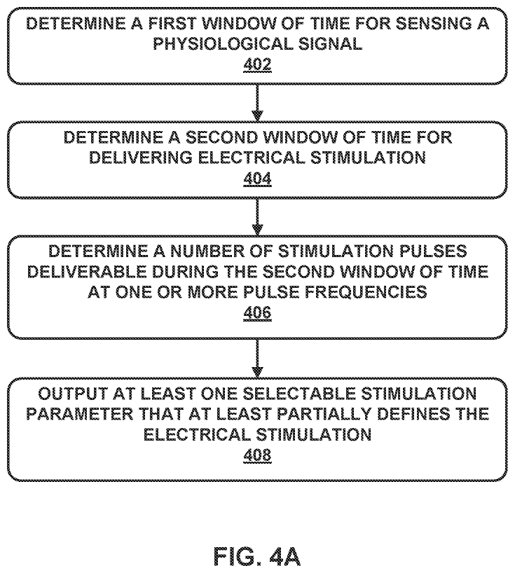

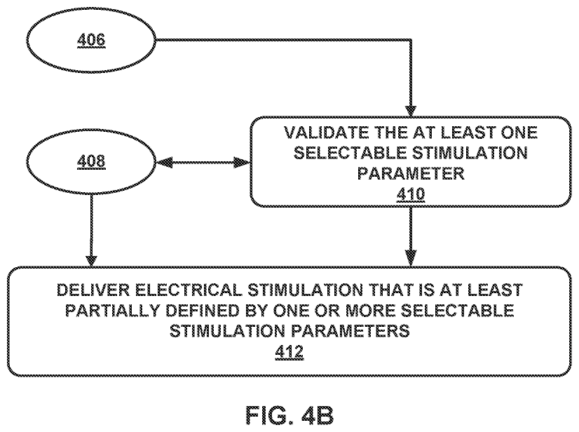

The techniques herein describe identifying additional pulse frequencies or patterns that can be delivered within certain periods of time, such as a therapy window. In general, processing circuitry 306 of programmer 150 may perform the processes described in FIGS. 4A-11 herein. However, other devices, or combination of devices, may perform these functions in other examples.

FIG. 4A is a flow diagram of an example method of parameter selection for electrical stimulation therapy based on an available sensing window and stimulation window. The example of FIG. 4A, processing circuitry 306 determines a first window of time for sensing a physiological signal (402). This first window may be the duration of time needed to sense a potential ECAP or other signal. Processing circuitry 306 then determines a second window of time for delivering electrical stimulation (404). This second window of time may be the window of time available between consecutive sensing windows. In other words, the second window may be the duration of time available to the system to deliver one or more stimulation pulses before the next sensing window.

Processing circuitry 306 can then determine the number of stimulation pulses that can be deliverable during the second window of time (e.g., the stimulation window) at one or more pulse frequencies (406). The number of pulses may be calculated for only one frequency, or may be calculated for two or more different frequencies, so that processing circuitry 306 can determine the number of pulses that can fit at the respective frequency. Processing circuitry 306 can then output at least one selectable stimulation parameter that at least partially defines the electrical stimulation to be delivered (408). The selectable stimulation parameter may be a value for a parameter such as frequency, pulse width, or even the number of consecutive pulses that can be delivered between consecutive sensing windows. Processing circuitry 306 may control a user interface to display these selectable options to a user or automatically select one of the options in order to satisfy stimulation criteria such as therapy efficacy, power consumption, etc.

FIG. 4B is a flow diagram of an example method of parameter selection for electrical stimulation therapy, further comprising validating the selectable stimulation parameters and delivering an electrical stimulation therapy at least partially defined by the selectable stimulation parameters. Reference numerals in circles herein refer to other steps or portions of the processes in other FIGS. FIG. 4B provides additional steps that may be added to the process of FIG. 4A ins some examples.

In the example of FIG. 4B, processing circuitry 306 may move from either of steps 406 or 408 in FIG. 4A to the validation step. In this step, processing circuitry 306 validates the at least one selectable stimulation parameter (410). For example, processing circuitry 306 may calculate the number of pulses at the selected frequency and confirm that the selected frequency can be used to deliver stimulation pulses within the available stimulation window. Then, processing circuitry 306 can control IMD 110 to deliver electrical stimulation that is at least partially defined by one or more selectable stimulation parameters (412). In the example of FIG. 4B, processing circuitry 306 may validate the selectable stimulation parameter after steps 406 and/or 408. In other words, processing circuitry 306 may validate the selectable parameter prior to the parameter being selected and/or after the parameter is selected for delivery of stimulation.

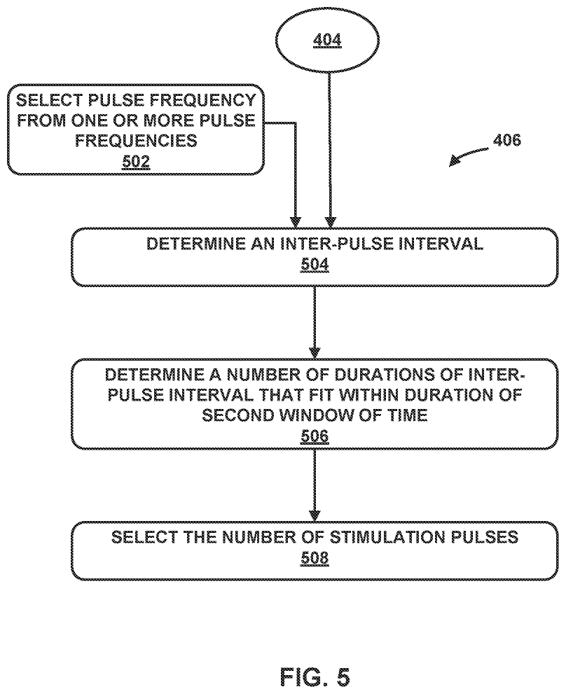

FIG. 5 is a flow diagram of an example method of determining the selectable stimulation parameters of FIG. 4A. The example of FIG. 5 may thus provide one example of more detailed processes of step 406 from FIG. 4A. Processing circuitry 306 may receive selection of a pulse frequency of one or more pulse frequencies from a user (e.g., via a user interface) or automatically select the pulse frequency (502). Based on the pulse frequency, processing circuitry 306 determines an inter-pulse interval (e.g., the period of time between consecutive pulses) for the stimulation (504). Then, processing circuitry 306 determines a number of durations of inter-pulse intervals that can fit within the duration of the second window of time (e.g., the stimulation window) (506). Using the number of inter-pulse intervals that fit, processing circuitry 306 determines the number of stimulation pulses that fit within the second window and with the number of inter-pulse intervals appropriate for the selected frequency (508). Processing circuitry 306 may perform the process of steps 504, 506, and 508 for multiple frequencies in some examples.

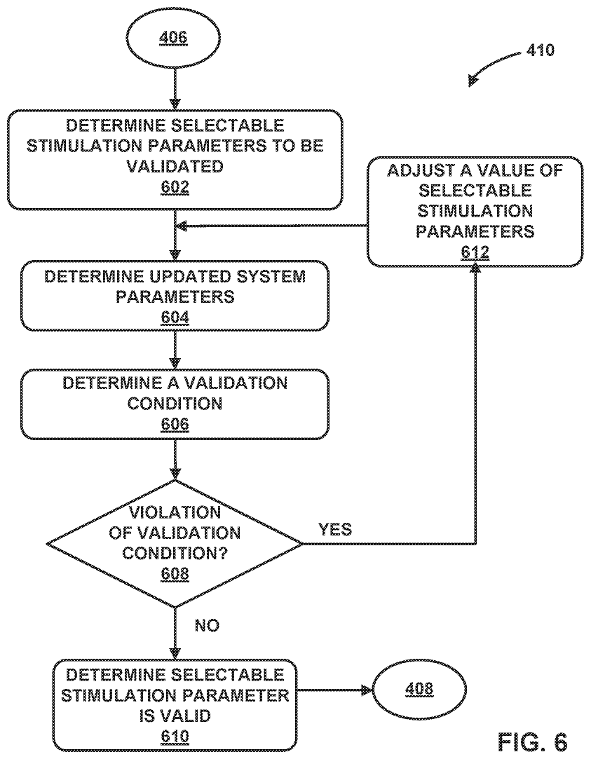

FIG. 6 is a flow diagram of an example method of validating the selectable stimulation parameters of FIG. 4B. If a parameter would violate one or more conditions, then that parameter is not used for programming options. In the example of FIG. 6, processing circuitry 306 receives the parameters from step 406 and then determines which selectable stimulation parameters that are to be validated (602). Processing circuitry 306 then determines the updated system parameters that correspond to the stimulation parameters to be validated (604). Then, processing circuitry 306 determines one or more validation conditions for the stimulation parameters (606) and determines if the stimulation parameters violate any of the validation conditions. Example validation conditions may include whether or not recharge phases interfere with a sensing window, a minimum number of stimulation pulses within the stimulation window to establish the selected frequency, or any other such conditions.

If the stimulation parameters violate any validation condition (“YES” branch of block 608), processing circuitry 306 then adjusts one or more values of the selectable stimulation parameters in order to achieve validation (612). If processing circuitry 306 determines that the stimulation parameters meet the validation conditions (“NO” branch of block 608), processing circuitry 306 determines that the selectable stimulation parameter(s) is valid (610) before moving to step 408.

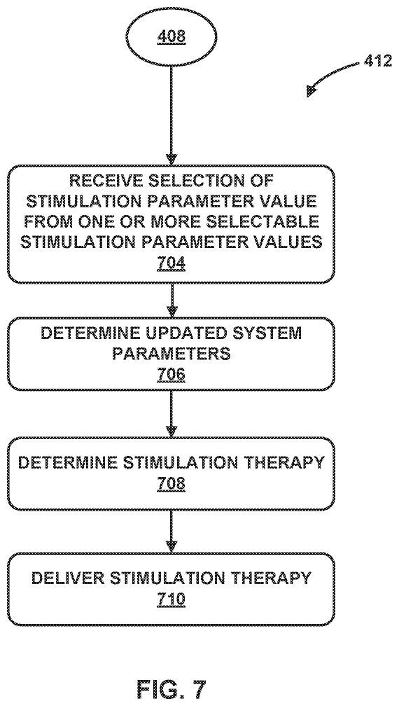

FIG. 7 is a flow diagram of an example process 412 of delivering stimulation therapies shown in FIG. 4B, for example. In the example of FIG. 7, processing circuitry 306 receives the selection of stimulation parameter values from one or more selectable stimulation parameter values from step 408, for example (704). In some examples, the selected parameter values may define the number of stimulation pulses or processing circuitry 306 may separately determine the number of stimulation pulses for each stimulation window. Then, processing circuitry 306 determines the updated system parameters that define stimulation therapy (708). Processing circuitry 306 then determines the stimulation therapy (708) and controls IMD 110 to deliver stimulation therapy (710).

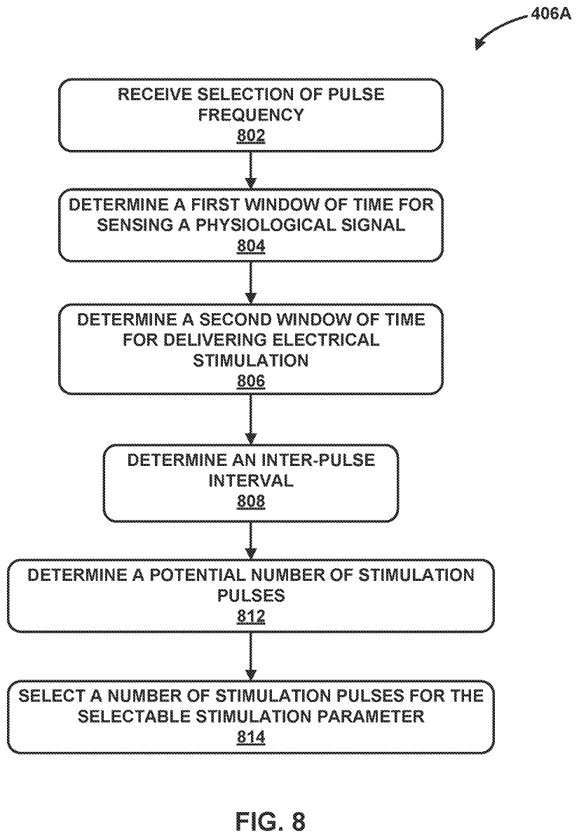

FIG. 8 is a flow diagram of an example method of determining a selectable stimulation parameter of FIG. 4A based on a selected therapy frequency. The process 406A of FIG. 8 may be an example of process 406 of FIG. 4A. In the example of FIG. 8, processing circuitry 306 receives a selection of pulse frequency (802). This selection may be via a user interface or by processing circuitry 306 itself. Processing circuitry 306 then determines a first window of time (e.g., a sensing window) for sensing a physiological signal (804). Next, processing circuitry 306 also determines a second window of time (e.g., a stimulation window) for delivering electrical stimulation (806). This second window of time may be the period of time between consecutive sensing windows that is available to deliver stimulation pulses.

Based on the selected pulse frequency, processing circuitry 306 determines an inter-pulse interval which is the period of time from the end of one pulse to the beginning of the next pulse (808). Using this inter-pulse interval, processing circuitry 306 determines a number of stimulation pulses that can be delivered within the second window of time (812). Using the number of pulses, processing circuitry 306 selects a number of stimulation pulses that will be delivered for the selectable stimulation parameter, such as the selected pulse frequency (814).

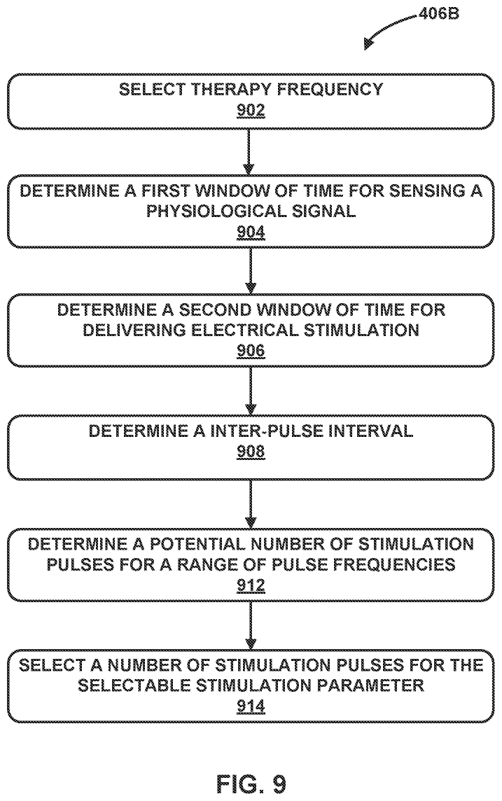

FIG. 9 is a flow diagram of an example method of determining a selectable stimulation parameter of FIG. 4A based on a range of therapy frequencies. The example of FIG. 9 is similar to the example of FIG. 8, but processing circuitry 306 can determine the number of stimulation pulses for a range of different pulse frequencies. The process 406B of FIG. 9 may be an example of process 406 of FIG. 4A. In the example of FIG. 9, processing circuitry 306 receives a selection of pulse frequency or pulse frequencies (902). This selection may be via a user interface or by processing circuitry 306 itself. Processing circuitry 306 then determines a first window of time (e.g., a sensing window) for sensing a physiological signal (904). Next, processing circuitry 306 also determines a second window of time (e.g., a stimulation window) for delivering electrical stimulation (906). This second window of time may be the period of time between consecutive sensing windows that is available to deliver stimulation pulses.

Based on the selected pulse frequency, processing circuitry 306 determines an inter-pulse interval which is the period of time from the end of one pulse to the beginning of the next pulse (908). Using this inter-pulse interval, processing circuitry 306 determines a number of stimulation pulses that can be delivered within the second window of time for each pulse frequency of the range of different pulse frequencies (912). Fewer number of pulses may fit within the second window for lower frequencies compared with greater number of pulses that may fit within the second window for higher frequencies. Using the number of pulses, processing circuitry 306 selects a number of stimulation pulses that will be delivered for the selectable stimulation parameter, such as the selected pulse frequency or multiple selected pulse frequencies (914).

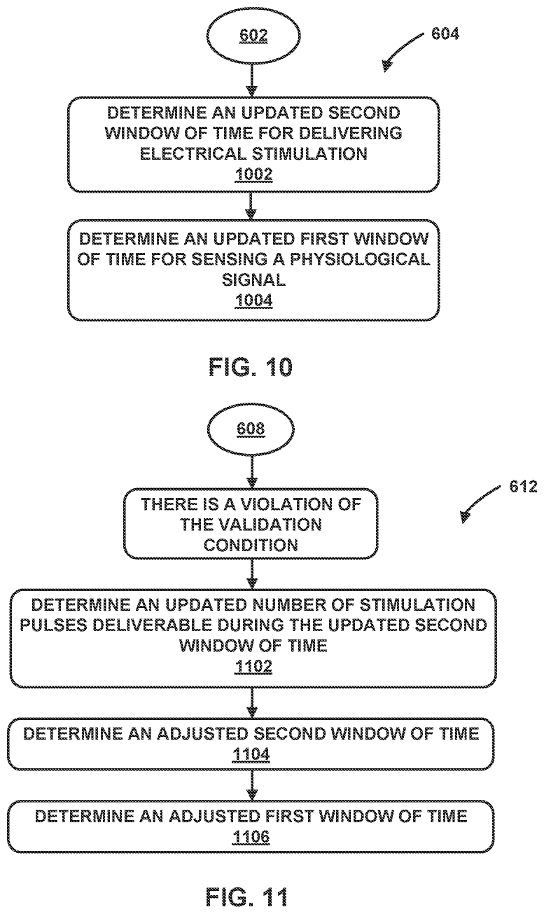

FIG. 10 is a flow diagram of an example method of determining updated system parameters of block 604 of FIG. 6, such as determining an updated window of time for delivering stimulation or sensing a physiological signal. As shown in the example of FIG. 10, processing circuitry 306 receives the selectable parameter to be validated in step 602 and then determines an updated second window of time for delivering electrical stimulation (1002). Then, processing circuitry 306 can determine an updated first window of time for sensing a physiological signal (1004). Using these first and second windows of time, processing circuitry 306 can control IMD 110 to deliver electrical stimulation and sense physiological signals from the patient.

FIG. 11 is a flow diagram of an example method of adjusting a value of the selectable stimulation parameters of block 612 of FIG. 6. As shown in the example of FIG. 11, processing circuitry 306 determines that the selectable parameter values violate a validation condition from step 608 and then determines an updated number of stimulation pulses deliverable during the updated second window of time (1102). Then, processing circuitry 306 determines an updated second window of time (1104) and an adjusted first window of time (1106) that may be appropriate for the updated number of stimulation pulses.

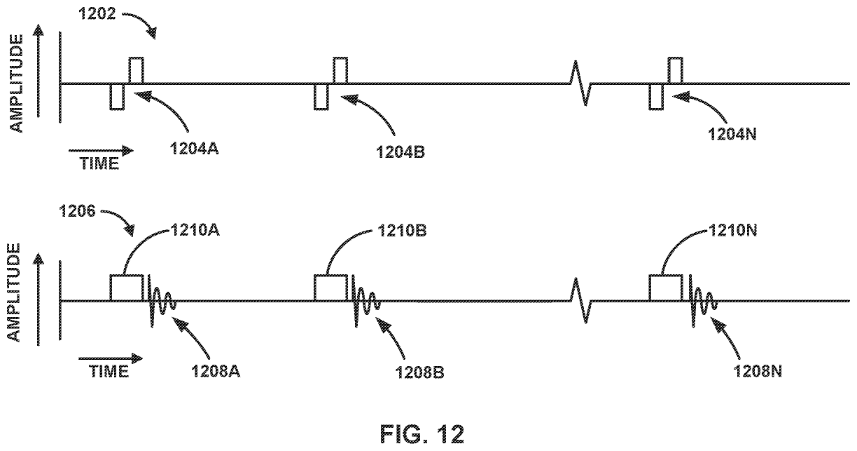

FIG. 12 is a timing diagram illustrating an example of evoked compound action potential (ECAP)-control intervals of control pulses and respective sensed ECAPs. For example, FIG. 12 is described with reference to IMD 110 of FIG. 2. As illustrated, the timing diagram includes first channel 1202, a plurality of control pulses 1204A-1204N (collectively “control pulses 1204”), second channel 1206, a plurality of respective ECAPs 1208A-1208N (collectively “ECAPs 1208”), and a plurality of stimulation interference signals 1210A-1210N (collectively “stimulation interference signals 1210”). In the example of FIG. 12, control pulses 1204 may also provide therapy to the patient and informed pulses are not necessary for therapy.

First channel 1202 is a time/voltage (and/or current) graph indicating the voltage (or current) of at least one electrode of electrodes 224, 226. In one example, the stimulation electrodes of first channel 1202 may be located on the opposite side of the lead as the sensing electrodes of second channel 1206. Control pulses 1204 may be electrical pulses delivered to the spinal cord of the patient by at least one of electrodes 224, 226, and control pulses 1204 may be balanced biphasic square pulses with an interphase interval. In other words, each of control pulses 1204 are shown with a negative phase and a positive phase separated by an interphase interval. For example, a control pulse 1204 may have a negative voltage for the same amount of time and amplitude that it has a positive voltage. It is noted that the negative voltage phase may be before or after the positive voltage phase. Control pulses 1204 may be delivered according to instructions stored in IMD 110, and may be updated according to user input via an external programmer and/or may be updated according to a signal from sensor(s) 212. In one example, control pulses 1204 may have a pulse width of less than approximately 300 microseconds (e.g., the total time of the positive phase, the negative phase, and the interphase interval is less than 300 microseconds). In another example, control pulses 1204 may have a pulse width of approximately 100 microseconds for each phase of the bi-phasic pulse. As illustrated in FIG. 12, control pulses 1204 may be delivered via one or more electrodes that deliver or sense signals corresponding to channel 1202. Delivery of control pulses 1204 may be delivered by leads 222 in a guarded cathode electrode combination. For example, if leads 222 are linear 8-electrode leads, a guarded cathode combination is a central cathodic electrode with anodic electrodes immediately adjacent to the cathodic electrode. For some patients, control pulses 1204 may sufficiently provide therapy that treats the condition and/or symptoms of the patient. Therefore, additional informed pulses may not be needed for these patients or for at least some aspect of the therapy for these patients.

Second channel 1206 is a time/voltage (and/or current) graph indicating the voltage (or current) of at least one electrode of electrodes 224, 226. In one example, the electrodes of second channel 1206 may be located on the opposite side of the lead as the electrodes of first channel 1202. ECAPs 1208 may be sensed at electrodes 224, 226 from the spinal cord of the patient in response to control pulses 1204. ECAPs 1208 are electrical signals which may propagate along a nerve away from the origination of control pulses 1204. In one example, ECAPs 1208 are sensed by different electrodes than the electrodes used to deliver control pulses 1204. As illustrated in FIG. 4A, ECAPs 1208 may be recorded on second channel 1206.

Stimulation interference signals 1210A, 1210B, and 1210N (e.g., the artifact of the stimulation pulses) may be sensed by leads 222 and may be sensed during the same period of time as the delivery of control pulses 1204. Since the interference signals may have a greater amplitude and intensity than ECAPs 1208, any ECAPs arriving at IMD 110 during the occurrence of stimulation interference signals 1210 may not be adequately sensed by sensing circuitry 206 of IMD 110. However, ECAPs 1208 may be sufficiently sensed by sensing circuitry 206 because each ECAP 1208 falls after the completion of each control pulse 1204. As illustrated in FIG. 12, stimulation interference signals 1210 and ECAPs 1208 may be recorded on channel 1206. The time period required to deliver a control pulse 1204 and corresponding ECAP 1208 may correspond to the sensing window. The therapy window may run from an ECAP 1208 to the next control pulse 1204 in time.

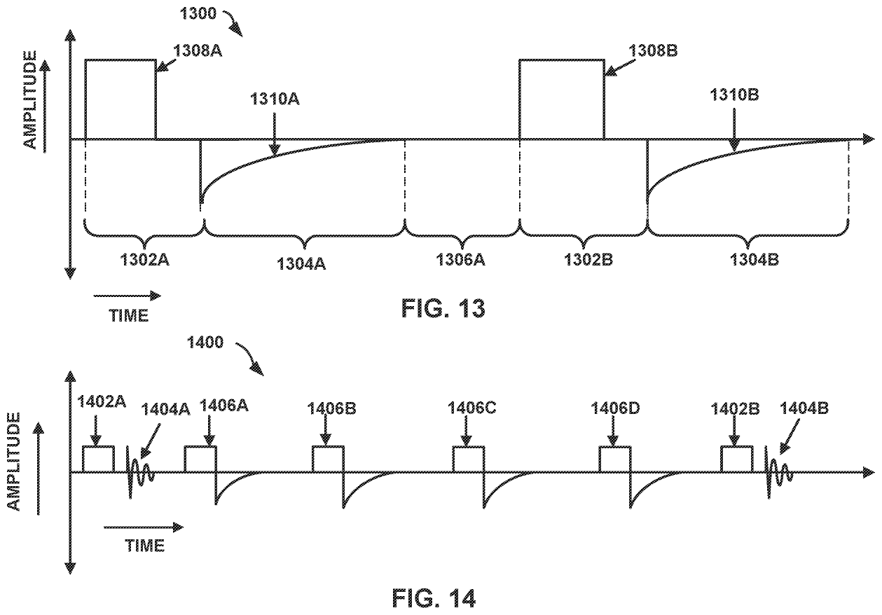

FIG. 13 is a timing diagram illustrating an example of stimulation pulses and accompanying phases of an inter-pulse interval. In the example of FIG. 13, timing diagram 1300 illustrates that each stimulation pulse may include a stimulation phase 1308 (such as phases 1308A and 1308B) and a following recharge phase (such as recharge phase 1310A and 1310B). Recharge phases 1310 are shown as passive recharge phases, but may be active recharge phases in other examples. Active recharge phases may form part of a biphasic stimulation pulse. In this manner, the stimulation phase 1308 corresponds to a stimulation time 1302 (i.e., 1308A and 1308B correspond to 1302A and 1302B, respectively). Similarly, the recharge phase 1310 corresponds to recharge time 1304 (i.e., 1310A and 1310B correspond to 1304A and 1304B, respectively). Inter-pulse interval 1306A is shown as the time period between adjacent recharge phases and the next stimulation phase.

FIG. 14 is a timing diagram illustrating an example of control pulses 1402, respective sensed ECAPs 1404, and stimulation pulses 1406 between adjacent ECAP-control intervals. As shown in the example of FIG. 14, timing diagram 1400 illustrates the time available in the therapy window within which stimulation pulses 1406 (e.g., 1406A-1406D) can be delivered. In other words, 4 stimulation pulses 1406 can be delivered between the sensing window that ends with ECAP signal 1404A and the sensing window that starts with control pulse 1402B. In some examples, the last of stimulation pulses 1406 can be changed to include an active recharge phase that completes in less time than the passive recharge phase in order to fit that stimulation pulse within the therapy window prior to the next control pulse 1402B. Such a change may be an example of an updated stimulation parameter in order for the number of pulses to not violate the validation conditions.

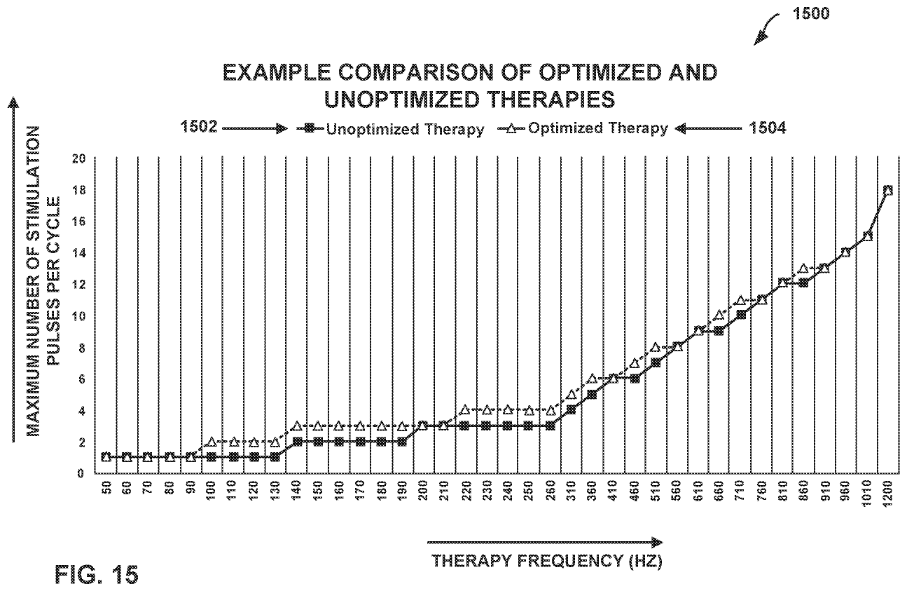

FIG. 15 is a graph illustrating the electrical stimulation options for stimulation therapy at varying therapy frequencies before and after application of the analysis described herein. As shown in example graph 1500 of FIG. 15, the techniques described herein for analyzing the therapy window for additional pulses that could be delivered. These additional pulses may enable a larger range of pulse frequencies to be used for stimulation therapy. For example, this analysis may enable higher frequencies for the same pulse width and therapy window. For example, adding an extra pulse can enable the system deliver pulses at a frequency of 100 Hz or 110 Hz with 2 pulses per cycle instead of needing to deliver at a rate of 140 Hz before 2 pulses would fit per cycle.

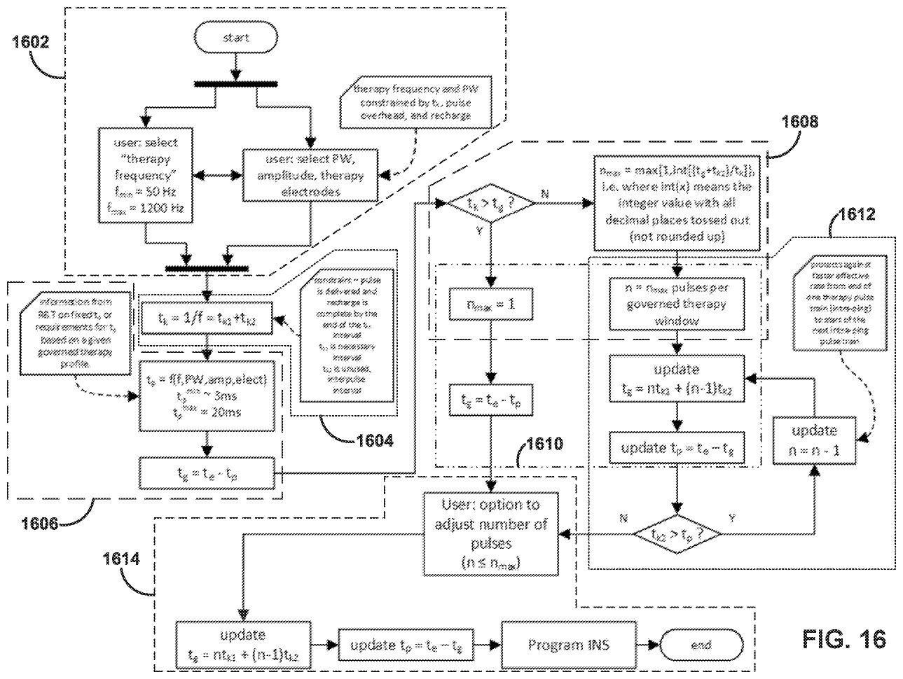

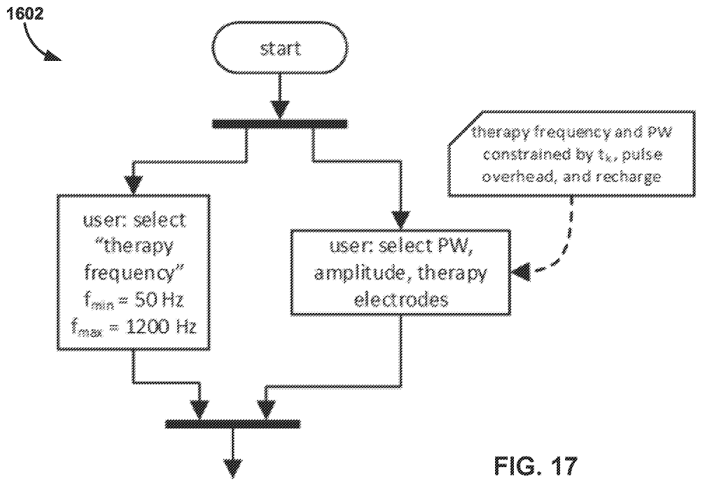

FIG. 16 is a flow diagram of an example method for determining the number of stimulation pulses that can be delivered between sensing windows. The example of FIG. 16 may be one example technique of the various techniques described herein. FIGS. 17-23 are flow diagrams illustrating portions 1602, 1604, 1606, 1608, 1610, 1612, and 1614 of the method of FIG. 16 and will be described below. Processing circuitry 306 will be described as one example, but any processing circuitry, device, system, or distributed system, may be used in other examples.

The example technique of FIG. 16 illustrates how a stimulation window (or a therapy window that may include stimulation pulses) can be effectively utilized to provide additional programming flexibility on available user selectable therapy frequencies and a higher upper bound on the therapy stimulation dose that can be delivered within the stimulation window. For example, the technique of FIG. 16 can be used to maximize the available therapy (e.g., the number of stimulation pulses) within the duration of the stimulation window or between sensing windows. However, it is noted that processing circuitry 306 can be configured to use less than the maximum number of pulses, depending on clinical or patient needs. Although various parameter values are described for illustrative purposes, other values and/or parameters may be used in other examples. Control pulses may be provided to elicit detectable ECAP signals (or other physiological signals), and informed pulses may be adjusted based on the ECAP signals and be configured to contribute to the therapy of the patient.

As shown in portion 1602 of FIGS. 16 and 17, once the method is started, the user selects the therapy frequency (e.g., the pulse frequency) and stimulation pulse width for the informed stimulation pulses. Processing circuitry 306 may receive this selection via a user interface. The user is prompted to select a therapy frequency (f) and pulse width (PW) for the individual pulses in the governed therapy interval (e.g., the informed pulses of the stimulation window). There may be limits to the available frequencies and the pulse width (duration) of the stimulation pulses. In this example, the minimum and maximum therapy frequencies are constraints on the user selections in our specific offering (such as a minimum of 50 Hz and a maximum of 1,200 Hz), but the specific values in this example are not constraints on any general stimulation system. In general, the minimum frequency may coincide with the frequency of the sensing windows in the closed-loop algorithm, but options could be made to run the informed stimulation pulse patterns at lower frequencies. However, these lower frequencies may have an integer multiple of the sensing window frequency in order to prevent therapy jitter and/or facilitate ease of implementation. Once the user selects a compatible pairing of therapy frequency and pulse width, processing circuitry 306 proceeds to the next step where various parameters are calculated.

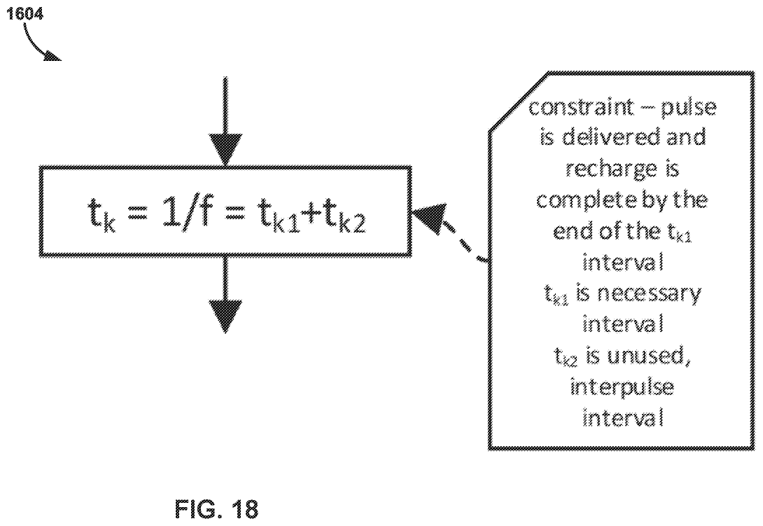

Next, processing circuitry 306 can calculate various time intervals for the algorithm. As shown in portion 1604 of FIGS. 16 and 18, once the therapy frequency f is selected, processing circuitry 306 can calculate the time period (tk) between each subsequent therapy pulse as the inverse of the frequency. Likewise, at this point two other parameters can be determined by processing circuitry 306. In this example, tk1 is the minimum time period necessary to deliver a single therapy pulse (e.g., an informed pulse) and provide adequate time to recover, through active or passive recharging, from the delivery of that pulse. The value of tk1 using passive recharge is approximately 4-5 times the therapy pulse width, i.e. 4×PW to 5×PW in some examples. In some examples processing circuitry 306 may more accurately calculate the recharge phase time that is needed, but processing circuitry 306 can assume 5×PW as the upper bound for tk1 to simplify the rest of the calculations. As an example, if a 200 microsecond (μs) PW is desired, the tk1=5PW=1 millisecond (ms). For active recharge, the value of tk1 should be shorter. This shorter recharge phase may permit additional pulses in the governed window (e.g., the stimulation window). The parameter tk2 is the time that is added after tk1 (where the pulse is delivered and recovered) in order to meet the total time (tk) used to adequately space out the therapy pulses such that the therapy frequency (f) is achieved.

Continuing with the example of a 200 μs pulse width with a calculated tk1=1 ms, if the desired therapy frequency f=1 kHz, then tk=1/f=1 ms. In this case tk2=tk−tk1=0 ms, so this example is at a maximum frequency scenario. As another example with the calculated tk1=1 ms, if the desired therapy frequency f=200 Hz, then tk=5 ms and tk2=tk−tk1=5 ms−1 ms=4 ms. The time period of tk2=4 ms is not needed for any purpose other than to adequately space out pulses while the tk1 value is used to deliver and recover from the delivered pulse. This differentiation between tk1 and tk2 is useful for maximizing the possible number of pulses provided by the system within a single stimulation window, especially at lower therapy frequencies. More sophisticated techniques can be used to determine tk1 for a given PW to further optimize pulse efficiency. Typically, when processing circuitry 306 determines that the maximum frequency supports a given pulse width, then the tk1(PW)=1/fmax. For example, a system in which a 200 μs pulse can be supported at any therapy frequency up to and including 1,200 Hz. Therefore, a 200 μs pulse width requires (1/1200=833.3 μs). Likewise a 700 μs pulse width can be supported at a frequency of 350 Hz or lower making tk1 (PW=700 μs)=1/350 Hz=2.86 ms.

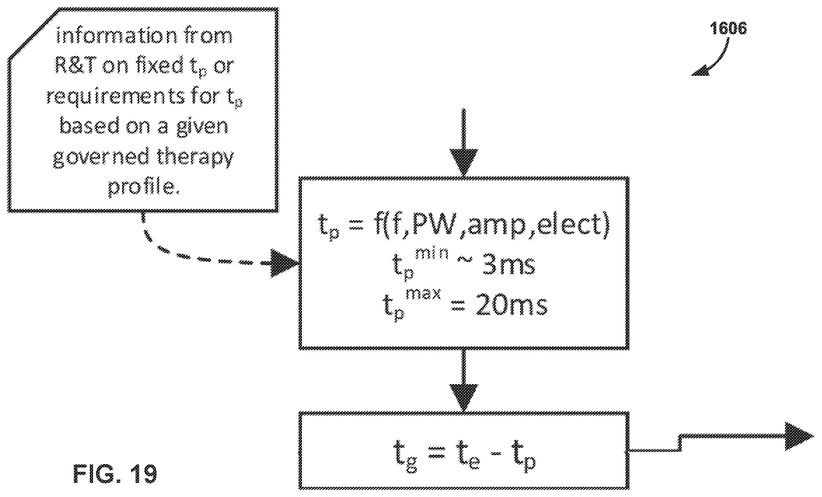

As shown in portion 1606 of FIGS. 16 and 19, processing circuitry 306 can calculate an initial estimate of the governed therapy window (e.g., the stimulation window) duration. The sensing window and respective measurements may be designed to operate at a certain rate. In one example, the rate is planned to be at 50 Hz, although this particular frequency may be different, higher or lower, in other examples. At this rate of 50 Hz, the interval between consecutive sensing windows, te=20 ms, indicates that each sensing window will occur 20 ms after the previous sensing window. This te value becomes a fixed value in this algorithm. Within this time period of te, both the ping/sense interval or sensing window (tp) and the governed interval or stimulation window (tg) have to fit within te such that te=tp+tg.

The ping/sense window needs an amount of time to prepare, deliver the ping (or control pulse), and sense an ECAP signal. This ping/sense window time is designated as tp. Depending on the desired governed therapy frequency, pulse width, and/or amplitude, the minimum tp value (tpmin) may require additional time for adequate ECAP sensing. This tpmin value for various stimulation profiles is determined by the design implementation and characterization. With a tpmin value (note tp>=tpmin), an initial estimate for the governed window duration (tg) can be calculated as: tg=te−tpmin. In one example where the ping/sense window requires the entire frame, i.e. tp=te, then by definition there is zero time for governed therapy (tg=0).

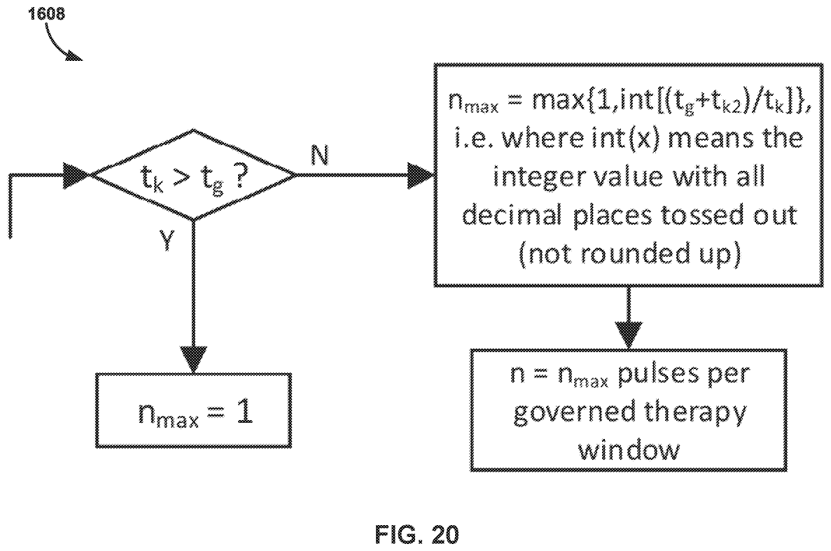

As shown in portion 1608 of FIGS. 16 and 20, processing circuitry 306 can calculate the maximum number of pulses that fit in the governed window. Now that an initial time window allocation for the governed therapy is calculated by processing circuitry 306, processing circuitry 306 can calculate the number of therapy pulses that can be placed in the governed window. In these calculations, the first governed pulse (e.g., an informed pulse) is delivered immediately after the completion of the ping/sense window, but processing circuitry 306 may control the first governed pulse to be delivered after some delay in other examples.

The governed pulse spacing (tk) is compared by processing circuitry 306 to the available time for the governed window (tg). If tk>tg, then it is not possible to put more than one therapy pulse in the governed frame, so the maximum number of pulses (nmax=1). For example, if a therapy frequency of 60 Hz is desired (tk=1/f=16.67 ms) and tg was calculated to be 15 ms, then processing circuitry 306 can only place one stimulation pulse in the governed window. In actual implementation, a frequency such as 60 Hz in this example may not be permitted because a validation condition may require two or more pulses within each governed window, but the example serves an instructive purpose.

If this criterion is not violated, then processing circuitry 306 performs another calculation. As shown in portion 1608, processing circuitry 306 calculates nmax as the maximum of either 1 or the integer value of (tg+tk2)/tk. This calculation considers the fact that the tk2 value is just used as time to space out pulses and is not needed for any other purpose than to space out governed therapy pulses. This is in contrast to the tk1 value which is needed to both deliver and recover from the delivered pulse. Now, there is an estimate for the total number of governed therapy pulses that can fit into the governed window.

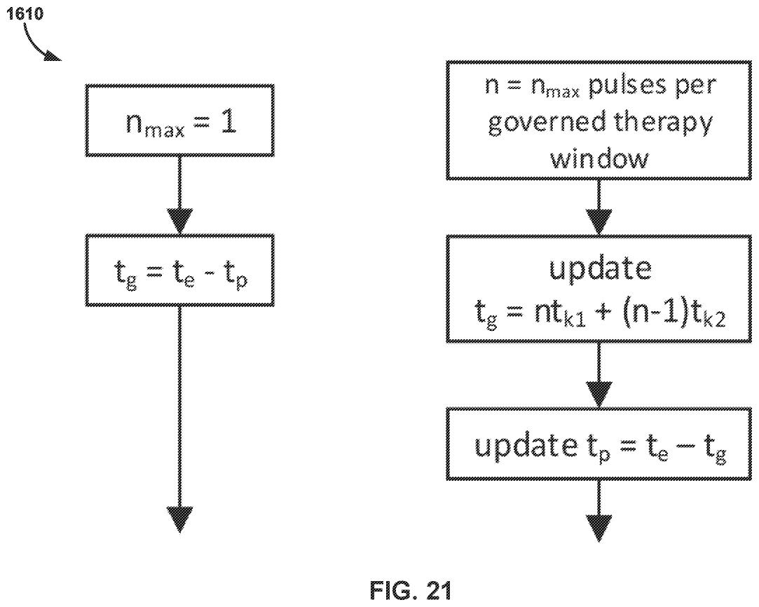

As shown in portion 1610 of FIGS. 16 and 21, processing circuitry 306 can calculate the actual time consumed in the delivery of n governed pulses. Processing circuitry 306 can calculate the time necessary to deliver n pulses with a minimum delivery/recovery time of tk1 per pulse as tg=ntk1+(n−1)tk2. This effectively permits the n pulses to be placed in the governed window with recognition that the final time interval of tk2 is not necessary since there will be no more governed therapy pulses within that particular governed window period. Therefore, the last tk2 time period is not needed to space out the next pulse. This is the reason for the calculations of tk1 and tk2 in the previous step. If tk1 and tk2 were lumped together and simply treated as requiring an amount of time equal to tk, then some available pulse trains would become unavailable by effectively limiting the number of pulses to 1, and processing circuitry 306 would lose the opportunity to satisfy some desired therapy frequencies. This treatment of tk1 and tk2 as separate time periods can permit additional pulses to be fit into the governed window, making some lower therapy frequency choices available to the user and also providing an extra pulse in the governed window at even higher frequencies.

At this point, processing circuitry 306 can make a more accurate calculation of tg and determine an update to the time allocated for the ping/sense window (tp). However, one additional check can be performed to ensure that the time between the delivery of the final pulse (pulse n) of this governed window does not violate the therapy frequency when the first pulse (pulse 1) of the subsequent governed frame is delivered.

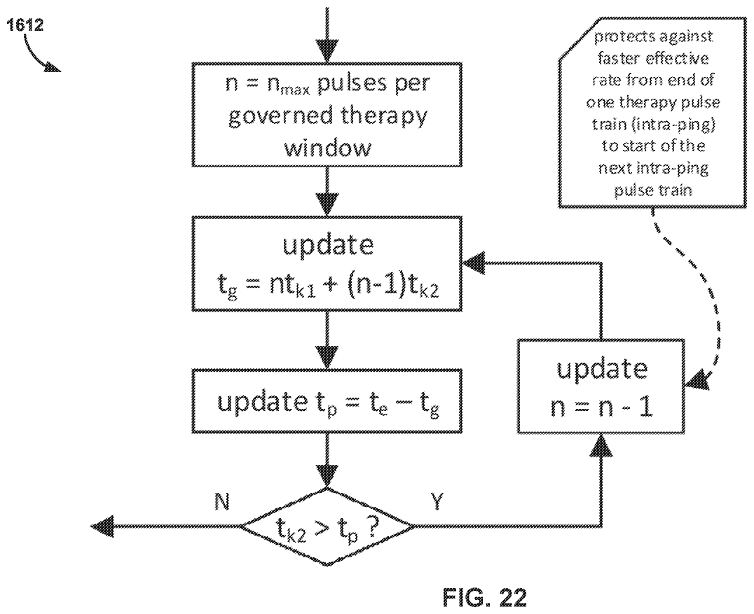

As shown in portion 1612 of FIGS. 16 and 22, processing circuitry 306 can check for governed window to governed window pulse timing. Recall that the first governed pulse is delivered immediately after the ping/sense interval is completed. The final check ensures that the updated ping/sense window (tp) is sufficiently long to prevent a violation of the therapy frequency from one governed window to the next. This is accomplished by processing circuitry 306 checking to see if the necessary time to space pulses (tk2) is shorter than the updated ping/sense window time (tp). If tk2<tp, then processing circuitry 306 determines that there is no violation. Otherwise, if tk2>tp, then one pulse needs to be removed to avoid this therapy frequency violation. Processing circuitry 306 then removes a pulse and recalculates the necessary governed window duration (tg) and updates the time allocated to the ping/sense window (tp), as illustrated in portion 1612 of FIG. 22.

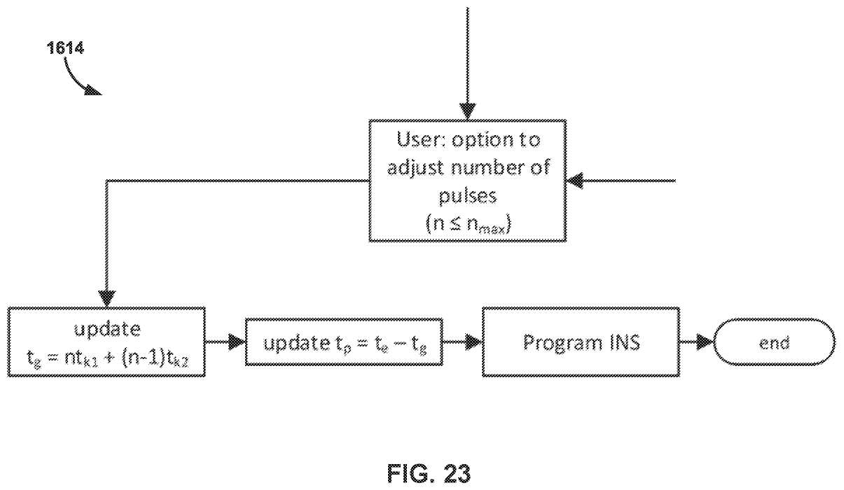

As shown in portion 1614 of FIGS. 16 and 23, processing circuitry 306 can perform final programming. Now the following are known: te, tg (based upon the calculated number of pulses that can be delivered), and tp (which is updated to be sufficiently long for the ping/sense interval as well as consuming the unused portion of tg which was originally assumed, but not needed after determining the time required to deliver n pulses). Processing circuitry 306 can add additional time from the updated tp to the pre-ping blanking time as that can improve sensing capabilities for ECAP measurements. Note that the updated tp>=tpmin is used in the initial assumption. This preserves the necessary time to adequately deliver a ping pulse (e.g., control pulse) and sense the ECAP from the patient. Now that the available time for ping/sense (tp) and the number of governed therapy pulses (n pulses requiring tk1 time to deliver and recover and tk2 time to space pulses to achieve the desired therapy frequency) is known, processing circuitry 306 can generate the entire waveform pattern and control IMD 110, for example, to deliver stimulation to the patient. This algorithm provides the maximum number of pulses available for use, but the maximum number does not need to be selected by the user or selected automatically by processing circuitry 306. For example, if 10 pulses are permitted within the stimulation window, a user could choose to deliver fewer pulses, such as five pulses, instead. In these instances, the updated allocated time for the desired number of pulses (where n≤nmax) in the governed therapy (tg) would be updated as tg=ntk1+(n−1)tk2 and the time allocated to ping/sense (tp) can be updated to capture the unused time via tp=te−tg. With all of the parameters now determined, processing circuitry 306 may store these parameters in memory and/or transfer the parameters to IMD 110 for controlling delivery of therapy to the patient and sensing information.

As discussed above, the identification of tk1 and tk2 time periods may provide for additional programming flexibility compared to limiting the governed therapies to a tk (1/f) time period per delivered pulse. In order to demonstrate this, we will contrast available frequencies, pulse widths, and maximum number of pulses using an example.

Example 1: frequency=120 Hz, PW=150 μs, te=20 ms, tpmin=5 ms, tg=20-5=15 ms, tk=1/f=1/120 Hz=8.33 ms. If tk1 and tk2 are ignored, then one pulse (nmax=1) can fit in the 15 ms tg window since the tk spacing is 8.33 ms. Placing two pulses would consume 16.67 ms which violates the allowable time for tg=15 ms. Since te=20 ms results in a 50 Hz therapy window, 120 Hz would not be permissible because only one pulse delivered every 20 ms (+=50 Hz) is allowed. With the 15 ms constraint, the minimum supportable frequency in this 15 ms governed therapy window would be 133.33 Hz.

However, if tk1 and tk2 are utilized, we have additional programming options and flexibility. For a 700 μs pulse, the tk1 value is approximately 2860 ms. This can be determined by recognizing that a 700 μs pulse width is supported by frequency up to 350 Hz, so 700 μs to 700 μs pulse spacing can be realized with a pulse-to-pulse spacing of about 2860 ms. So, in this example, tk1=2860 and tk2=8333 μs−2860 μs=5473 μs. Using our algorithm, nmax=2 for this 120 Hz therapy frequency. The resulting tg=11.19 ms and tp=8.81 ms.

The following figure illustrates a modest, but important, improvement in the maximum number of therapy pulses available at the different therapy frequencies (assuming a 200 μs pulse width in each instance). In some cases, there is little difference between the two methods, but the tk1 method permits an additional pulse from 100 to 130 Hz (inclusive), making these therapy choices selectable because the single pulse limitation with the less complex method effectively limits these therapy frequencies to be 50 Hz). Again, an additional pulse is available between 50-90 Hz (inclusive). At higher frequencies, the two methods begin to converge, although there are instances where the tk1 method provides a few more available pulses for the governed therapy window. These example frequencies that are made available are shown in optimized therapy line 1504 in graph 1500 of FIG. 15.

The example above in which the ping/sense window is delivered at a rate of 50 Hz is merely one example. Other lower or higher ping/sense window frequencies can be used in other examples. In addition, the preceding example indicated that excess time not utilized by the governed window (tg) is applied back to the pre-ping pulse window time (i.e., excess time added back to tp). However, processing circuitry 306 may automatically, or in response to user input, select different ways to incorporate the excess time not utilized by the governed window. In one example, processing circuitry 306 can apply excess tg time to additional passive recharge time on the end of the governed window. This may improve charge balancing after delivery of stimulation. In another example, processing circuitry 306 may apply excess tg time to increase the pre-ping quiet time. This increased time before delivering the ping or control pulse may improve the invoking and sensing of an ECAP signal. In another example, processing circuitry 306 may apply excess tg time to post-ping/pre-governed time in order to space out the governed pulses further from the ping/sense window and center the governed therapy more in the governed window. In another example, processing circuitry 306 may use the excess tg time to blank the system during this time with no additional functionality.

In some examples, it may be beneficial, from a subsequent sensing function perspective, to utilize active recharge on the final pulse of the governed pulse train. Active recharge would enable the system to more efficiently remove, or balance, residual charge in the system and provide a more stable environment for sensing during the next sensing window. Although active recharge uses more power than passive recharge, applying the active recharge to only the final pulse within the stimulation window may help provide greater charge recovery without a significant power consumption requirement as compared to using active recharge on every stimulation pulse. The final pulse in the governed waveform train with active recharge could have an active recharge phase with a slightly longer charge recovery capability, e.g. longer pulse width, in order to collect or recover charge from the last pulse as well as previous governed therapy pulses.

The following examples are described herein.

Example 1. A device comprising: processing circuitry configured to: determine a first window of time to sense a physiological signal; determine, based on the first window of time, a second window of time for delivering electrical stimulations; determine, based on a duration of the second window of time, a number of stimulation pulses deliverable during the second window of time at one or more pulse frequencies; and output, based on the number of stimulation pulses deliverable during the second window of time, at least one selectable stimulation parameter that at least partially defines the electrical stimulation, wherein the second window of time is adjacent to the first window of time.

Example 2. The device of example 1, wherein the processing circuitry configured to determine the number of stimulation pulses that are deliverable comprises processing circuitry that, for each frequency of the one or more pulse frequencies: determines a duration of an inter-pulse interval; determines a number of durations of the inter-pulse interval that fit within the duration of the second window of time; and selects the number of stimulation pulses less than or equal to the number of durations of the inter-pulse interval that fit within the duration of the second window of time.

Example 3. The device of example 2, wherein the inter-pulse interval comprises a stimulus phase, a recharge phase, and an idle phase.

Example 4. The device of any of examples 1 through 3, wherein the physiological signal comprises one or more evoked compound action potential (ECAP) signals, wherein the first window of time comprises an ECAP control interval, and wherein the second window of time comprises a duration between two adjacent ECAP control intervals.

Example 5. The device of example 4, wherein the ECAP control interval comprises a control pulse configured to elicit an ECAP signal of the one or more ECAP signals.

Example 6. The device of any of examples 1 through 5, wherein the processing circuitry is further configured to validate the selectable stimulation parameter, wherein the processing circuitry validates the selectable stimulation parameter by iteratively determining a set of updated parameters, determining, based on the set of updated parameters a validation condition, determining a violation of the validation condition, and responsive to determining the violation of the validation condition, adjusting a value of the at least one selectable stimulation parameter.

Example 7. The device of example 6, wherein determining the set of update parameters comprises: determining, based on the number of stimulation pulses deliverable during the second window of time, an updated second window of time; and determining, based on the updated second window of time, an updated first window of time, and wherein determining the violation of the validation condition comprises determining that an idle phase of an inter-pulse interval is longer than the updated first window of time.

Example 8. The device of example 7, wherein adjusting the value of the selectable stimulation parameter comprises: determining, based on the number of stimulation pulses deliverable during the second window of time, an updated number of stimulation pulses deliverable during the updated second window of time; and determining a set of adjusted parameters, wherein determining the set of adjusted parameters comprises: determining, based on the updated number of stimulation pulses deliverable during the updated second window of time, an adjusted second window of time; and determining, based on the adjusted second window of time, an adjusted first window of time.

Example 9. The device of any of examples 1 through 8, wherein the processing circuitry is further configured to deliver the electrical stimulation, wherein the electrical stimulation is at least partially defined by the one or more selectable stimulation parameters.

Example 10. The device of example 9, wherein the processing circuitry controls an implanted medical device to deliver the electrical stimulation.

Example 11. The device of any of examples 1 through 10, wherein the processing circuitry is a medical device programmer comprising the processing circuitry.

Example 12. A method comprising: determining, by processing circuitry, a first window of time for sensing a physiological signal; determining, by the processing circuitry and based on the first window of time, a second window of time for delivering electrical stimulation; determining, based on a duration of the second window of time, a number of stimulation pulses deliverable during the second window of time at one or more pulse frequencies; and outputting, based on the number of stimulation pulses deliverable during the second window of time, at least one selectable stimulation parameter that at least partially defines the electrical stimulation, wherein the second window of time is adjacent to the first window of time.

Example 13. The method of example 12, wherein determining the number of stimulation pulses deliverable during the second window of time comprises, for each frequency of the one or more pulse frequencies: determining a duration of an inter-pulse interval; determining a number of durations of the inter-pulse interval that fit within the duration of the second window of time; and selecting the number of stimulation pulses less than or equal to the number of durations of the inter-pulse interval that fit within the duration of the second window of time.

Example 14. The method of example 13, wherein the inter-pulse interval comprises a stimulus phase, a recharge phase, and an idle phase.

Example 15. The method of any of examples 12 through 14, wherein the physiological signal comprises one or more evoked compound action potential (ECAP) signals, wherein the first window of time comprises an ECAP control interval, and wherein the second window of time comprises a duration between two adjacent ECAP control intervals.

Example 16. The method of example 15, wherein the ECAP control interval comprises a control pulse configured to elicit an ECAP signal of the one or more ECAP signals.

Example 17. The method of any of examples 12 through 16, further comprising validating, by the processing circuitry, the at least one selectable stimulation parameter, wherein validating the selectable simulation parameter comprises: iteratively determining a set of updated parameters; determining, based on the set of updated parameters, a validation condition; determining a violation of the validation condition; and responsive to determining the violation of the validation condition, adjusting a value of the at least one selectable stimulation parameter.

Example 18. The method of example 17, wherein determining the set of updated parameters comprises: determining, based on the number of stimulation pulses deliverable during the second window of time, an updated second window of time; and determining, based on the updated second window of time, an updated first window of time, wherein determining the violation of the validation condition comprises determining that an idle phase of an inter-pulse interval is longer than the updated first window of time.

Example 19. The method of example 18, wherein adjusting the value of the selectable stimulation parameter comprises: determining, based on the number of stimulation pulses deliverable during the second window of time, an updated number of stimulation pulses deliverable during the updated second window of time; and determining a set of adjusted parameters, wherein determining the set of adjusted parameters comprises: determining, based on the updated number of stimulation pulses deliverable during the updated second window of time, an adjusted second window of time; and determining, based on the adjusted second window of time, an adjusted first window of time.

Example 20. The method of example 17, wherein validating the selectable stimulation parameter further comprises: determining, based on a set of adjusted selectable stimulation parameters, an adjusted validation condition; and determining a violation of the adjusted validation condition, wherein determining the violation of the adjusted validation condition comprises determining that an idle phase of an inter-pulse interval is longer than an adjusted first window of time, and the set of adjusted selectable stimulation parameters comprise the adjusted first window of time.

Example 21. The method of any of examples 12 through 20, further comprising delivering, by processing circuitry, electrical stimulation, wherein the electrical stimulation is at least partially defined by the at least one selectable stimulation parameters.

Example 22. The method of any of examples 12 through 21, wherein the processing circuitry controls an implanted medical device to deliver the electrical stimulation.

Example 23. A system comprising: an implanted medical device; a medical device programmer; and processing circuitry configured to: determine a first window of time to sense a physiological signal; determine, based on the first window of time, a second window of time for delivering electrical stimulations; determine, based on a duration of the second window of time, a number of stimulation pulses deliverable during the second window of time at one or more pulse frequencies; and output, based on the number of stimulation pulses deliverable during the second window of time, at least one selectable stimulation parameter that at least partially defines the electrical stimulation, wherein the second window of time is adjacent to the first window of time.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

1. A device comprising:
processing circuitry configured to:
determine a first window of time to sense a physiological signal;
determine, based on the first window of time, a second window of time for delivering electrical stimulations;
determine, based on a duration of the second window of time, a number of stimulation pulses deliverable during the second window of time at one or more pulse frequencies; and
output, based on the number of stimulation pulses deliverable during the second window of time, at least one selectable stimulation parameter that at least partially defines the electrical stimulation,
wherein the second window of time is adjacent to the first window of time.

2. The device of claim 1, wherein the processing circuitry configured to determine the number of stimulation pulses that are deliverable comprises processing circuitry that, for each frequency of the one or more pulse frequencies:
determines a duration of an inter-pulse interval;
determines a number of durations of the inter-pulse interval that fit within the duration of the second window of time; and
selects the number of stimulation pulses less than or equal to the number of durations of the inter-pulse interval that fit within the duration of the second window of time.

3. The device of claim 2, wherein the inter-pulse interval comprises a stimulus phase, a recharge phase, and an idle phase.

4. The device of claim 1, wherein the physiological signal comprises one or more evoked compound action potential (ECAP) signals, wherein the first window of time comprises an ECAP control interval, and wherein the second window of time comprises a duration between two adjacent ECAP control intervals.

5. The device of claim 4, wherein the ECAP control interval comprises a control pulse configured to elicit an ECAP signal of the one or more ECAP signals.

6. The device of claim 1, wherein the processing circuitry is further configured to validate the selectable stimulation parameter, wherein the processing circuitry validates the selectable stimulation parameter by iteratively determining a set of updated parameters, determining, based on the set of updated parameters a validation condition, determining a violation of the validation condition, and responsive to determining the violation of the validation condition, adjusting a value of the at least one selectable stimulation parameter.

7. The device of claim 6, wherein determining the set of update parameters comprises:
determining, based on the number of stimulation pulses deliverable during the second window of time, an updated second window of time; and
determining, based on the updated second window of time, an updated first window of time, and
wherein determining the violation of the validation condition comprises determining that an idle phase of an inter-pulse interval is longer than the updated first window of time.

8. The device of claim 7, wherein adjusting the value of the selectable stimulation parameter comprises:
determining, based on the number of stimulation pulses deliverable during the second window of time, an updated number of stimulation pulses deliverable during the updated second window of time; and
determining a set of adjusted parameters, wherein determining the set of adjusted parameters comprises:
determining, based on the updated number of stimulation pulses deliverable during the updated second window of time, an adjusted second window of time; and
determining, based on the adjusted second window of time, an adjusted first window of time.

9. The device of claim 1, wherein the processing circuitry is further configured to deliver the electrical stimulation, wherein the electrical stimulation is at least partially defined by the one or more selectable stimulation parameters.

10. The device of claim 9, wherein the processing circuitry controls an implanted medical device to deliver the electrical stimulation.

11. The device of claim 1, wherein the device is a medical device programmer comprising the processing circuitry.

12. A method comprising:
determining, by processing circuitry, a first window of time for sensing a physiological signal;
determining, by the processing circuitry and based on the first window of time, a second window of time for delivering electrical stimulation;
determining, based on a duration of the second window of time, a number of stimulation pulses deliverable during the second window of time at one or more pulse frequencies; and
outputting, based on the number of stimulation pulses deliverable during the second window of time, at least one selectable stimulation parameter that at least partially defines the electrical stimulation, wherein the second window of time is adjacent to the first window of time.

13. The method of claim 12, wherein determining the number of stimulation pulses deliverable during the second window of time comprises, for each frequency of the one or more pulse frequencies:
determining a duration of an inter-pulse interval;
determining a number of durations of the inter-pulse interval that fit within the duration of the second window of time; and
selecting the number of stimulation pulses less than or equal to the number of durations of the inter-pulse interval that fit within the duration of the second window of time.

14. The method of claim 13, wherein the inter-pulse interval comprises a stimulus phase, a recharge phase, and an idle phase.

15. The method of claim 12, wherein the physiological signal comprises one or more evoked compound action potential (ECAP) signals, wherein the first window of time comprises an ECAP control interval, and wherein the second window of time comprises a duration between two adjacent ECAP control intervals.

16. The method of claim 15, wherein the ECAP control interval comprises a control pulse configured to elicit an ECAP signal of the one or more ECAP signals.

17. The method of claim 12, further comprising validating, by the processing circuitry, the at least one selectable stimulation parameter, wherein validating the selectable simulation parameter comprises: iteratively determining a set of updated parameters; determining, based on the set of updated parameters, a validation condition; determining a violation of the validation condition; and responsive to determining the violation of the validation condition, adjusting a value of the at least one selectable stimulation parameter.

18. The method of claim 17, wherein determining the set of updated parameters comprises: determining, based on the number of stimulation pulses deliverable during the second window of time, an updated second window of time; and determining, based on the updated second window of time, an updated first window of time, wherein determining the violation of the validation condition comprises determining that an idle phase of an inter-pulse interval is longer than the updated first window of time.

19. The method of claim 12, further comprising delivering, by processing circuitry, spinal cord stimulation to a patient, wherein the spinal cord stimulation is at least partially defined by the at least one selectable stimulation parameters.

20. A system comprising:
an implanted medical device;
a medical device programmer; and
processing circuitry configured to:
determine a first window of time to sense a physiological signal;
determine, based on the first window of time, a second window of time for delivering electrical stimulations;
determine, based on a duration of the second window of time, a number of stimulation pulses deliverable during the second window of time at one or more pulse frequencies; and
output, based on the number of stimulation pulses deliverable during the second window of time, at least one selectable stimulation parameter that at least partially defines the electrical stimulation, wherein the second window of time is adjacent to the first window of time.
